(19) United States
(12) Patent Application Publication
(10) Pub. No.: US 2022/0101334 A1
(43) Pub. Date: Mar. 31, 2022

(54) INDIVIDUALIZED CURRICULUM OF ENGAGEMENT GENERATION BASED ON USER INFORMATION

(71) Applicant: Transilio, Inc., Boise, ID (US)

(72) Inventors: Michael Boerner, Boise, ID (US); Roger Zanders, JR., Meridian, ID (US)

(73) Assignee: Transilio, Inc., Boise, ID (US)

(21) Appl. No.: 17/319,627

(22) Filed: May 13, 2021

Related U.S. Application Data
(63) Continuation of application No. 16/502,804, filed on Jul. 3, 2019, now abandoned.
Application No. 16/502,804 is a continuation of application No. 14/214,313, filed on Mar. 14, 2014, now abandoned.
(60) Provisional application No. 61/800,667, filed on Mar. 15, 2013.

(51) Int. Cl. G06Q 30/00; G16H 20/10; G06Q 40/08

(52) U.S. Cl. CPC G06Q 30/016; G06Q 40/08; G16H 20/10

(57) ABSTRACT

System and method for user engagement are provided. The methods may include receiving at one or more computing devices information about a user obtained at a point of interaction; generating using the one or more computing devices, based at least in part on the information, an individualized curriculum of engagement for the user; and delivering using the one or more computing devices the individualized curriculum of engagement to the user. The system may include an input computing device receiving information about a user at a point of interaction and sending the information to one or more servers; the one or more servers generating, based at least in part on the information, an individualized curriculum of engagement for the user and sending the individualized curriculum of engagement to a user computing device; and the user computing device receiving the individualized curriculum of engagement from the one or more servers.

### CROSS-REFERENCE TO RELATED PATENT APPLICATIONS

This application claims priority to U.S. Provisional Patent Application 61/800,667 filed Mar. 15, 2013, which is incorporated herein by reference in its entirety.

### BACKGROUND

For years, businesses have been struggling with how to engage their customers after they have made their purchases (physically or virtually) at a commerce location. This is particularly relevant in medical services and the retail environment. The lack of post-purchase engagement has resulted in non-adherence in the case of medications, returned merchandise in the case of physical goods, lack of loyalty in the case of services, and lost business opportunities associated with the lack of ongoing linkages. Post-purchase engagement would yield significant benefits to both service providers and the consumers themselves.

A specific case study involves consumer adherence to pharmaceutical medication. Nearly 50% of U.S. citizens take prescriptions, and nearly 3.7 billion prescriptions are written each year. Over 75% of Americans self-report that they do not take their medications as directed, and 33% never fill their prescriptions at all. This non-adherence has been costly through premature deaths, long-term sicknesses, and economic losses. Studies have suggested a lack of education (e.g., not understanding one's medical condition, the efficacy or side effects of a drug) and forgetfulness account for nearly 70% of the non-adherence problem. To address the non-adherence issue, the medics would like to spend more time educating their patients. It is also well documented that the “human-process bottleneck” in medicine is a reality; doctors do not have as much time as desired with patients, only a fraction of patients receive counseling from pharmacists and the patient “take-away” from their pharmacy visit consists of tiny print on a difficult to read drug-fact sheet.

Another case study involves the consumer adoption of home electronics. Millions of home-electronics are purchased worldwide, including personal computers. Many people struggle to learn how to use them resulting in low utilization, incorrect utilization, fatalities and a high rate of returns to the stores. The returns are expensive to the retailers. Retailers therefore acknowledge that post-purchase engagement with the consumer is crucial to the adoption of the technology, customer loyalty and overall economics. However, retailers have experienced a significant disconnect between the purchaser, the manufacturer, and the retailers themselves as most purchasers never provide explicit feedback on how they utilize the electronics they have purchased.

### SUMMARY

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

Accordingly, provided in one embodiment is a method. The method includes receiving at one or more computing devices information about a user obtained at a point of interaction. The method further includes generating, using the one or more computing devices, based at least in part on the information about the user, an individualized curriculum of engagement for the user. The method further includes delivering, using the one or more computing devices, the individualized curriculum of engagement to the user.

In another embodiment, a method is provided. The method includes receiving, using a computing device, information about a user at a point of interaction. The method further includes sending the information from the computing device to a webspace. The method further includes generating, based at least in part on the information in the webspace, an individualized curriculum of engagement for the user. The method further includes delivering the individualized curriculum of engagement to the user.

In another embodiment, a system for user engagement is provided. The system includes an input computing device configured to receive information about a user at a point of interaction and to send the information to one or more servers. The system further includes the one or more servers configured to generate, based at least in part on the information received from the input computing device, an individualized curriculum of engagement for the user and to send the individualized curriculum of engagement to a user computing device. The system further includes the user computing device configured to receive the individualized curriculum of engagement from the one or more servers.

### BRIEF DESCRIPTION OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally similar and/or structurally similar elements).

FIG. 1 is a schematic flow chart showing a personalized retail engagement pathway according to some embodiments.

FIG. 2 is a diagram showing various steps of a personalized retail engagement pathway according to some embodiments.

FIG. 3 is a system diagram showing elements in a system for user engagement according to some embodiments.

FIG. 4 is a flowchart showing a process for user engagement according to some embodiments.

FIG. 5 is a flowchart showing a process for user engagement according to some embodiments.

FIG. 6 is a flowchart showing a process for user engagement according to some embodiments.

FIG. 7 is a diagram showing elements in a system and movement of information in a process for user engagement according to some embodiments.

FIG. 8 is a diagram showing elements in a system and movement of information in a process for user engagement according to some embodiments.

FIGS. 9A and 9B together show a roles/permissions matrix according to some embodiments.

FIG. 10 shows a client site workflow according to some embodiments.

FIG. 11 shows an admin site workflow according to some embodiments.

FIG. 12 shows a data model according to some embodiments.

FIGS. 13A and 13B together show a roles/permissions matrix according to some embodiments.

FIG. 14 shows a client site workflow according to some embodiments.

FIG. 15 shows an admin site workflow according to some embodiments.

FIG. 16 shows an exemplary account creation interface according to some embodiments.

FIG. 17 shows an exemplary front end sign in interface according to some embodiments.

FIG. 18 shows an exemplary front end landing page interface according to some embodiments.

FIG. 19 shows an exemplary channel home page interface according to some embodiments.

FIG. 20 shows an exemplary show home page interface according to some embodiments.

FIG. 21 shows an exemplary admin dashboard interface according to some embodiments.

FIG. 22 shows an exemplary channel list interface according to some embodiments.

FIG. 23 shows an exemplary channel maintenance interface according to some embodiments.

FIG. 24 shows an exemplary channel maintenance interface according to some embodiments.

FIG. 25 shows an exemplary delivery settings interface according to some embodiments.

FIG. 26 shows an exemplary episode editing interface according to some embodiments.

FIG. 27 shows an exemplary episode interface according to some embodiments.

FIG. 28 shows an exemplary episode adding interface according to some embodiments.

FIG. 29 shows an exemplary system diagram according to some embodiments.

FIG. 30 shows an exemplary interaction diagram according to some embodiments.

FIG. 31 shows an exemplary set of software modules according to some embodiments.

FIG. 32 shows a data model according to some embodiments.

### DETAILED DESCRIPTION

Following below are more detailed descriptions of various concepts related to, and embodiments of, inventive individualized curriculum of engagement generation based on user information. It should be appreciated that various concepts introduced above and discussed in greater detail below may be implemented in any of numerous ways, as the disclosed concepts are not limited to any particular manner of implementation. Examples of specific implementations and applications are provided primarily for illustrative purposes.

In the following disclosure, some terms may be used with special meaning to the present embodiments. The term “consumer” is used. In some embodiments, a “consumer” is an all-inclusive definition of a person who receives a service, purchases a product, attends a course, and/or is involved in a commercial activity as a receiver of that commerce. The term “pathway” is used. In some embodiments, a “pathway” is a personalized engagement roadmap that is created algorithmically for a consumer to further enhance his/her knowledge about the product or service he/she has received. The term “purchasing data” is used. In some embodiments, “purchasing data” refers to the data involving the consumer's actions at the point of service provision or purchase. The term “promotional” is used with various other terms, such as “promotional video,” “promotional offer,” “promotional information,” etc. In some embodiments, “promotional” means something generally tending to promote the purchase, consumption, or other use of some product, service, or other item. The terms “computer,” “computing device,” and other similar variations are used. In some embodiments, these terms refer similarly to electronic computing devices. These may be implemented in various forms, including cellular telephones, smart phones, PDAs, tablet computers, laptop computers, desktop computers, and other forms. The terms “point of engagement” and “point of interaction are used. In some embodiments, these phrases refer similarly to a point wherein a consumer has some sort of interaction, engagement, transaction, etc. These phrases may refer to a point of sale in some embodiments. The terms “interactions” and “engagements” and other similar terms are used. In some embodiments, these terms are used similarly to refer to contact made by the system with a user in order to interact with the user and thereby engage the user. Other similar terms may be used to describe this action.

One embodiment provides a process and method of transferring consumer data from the retail environment to personalized platforms for the purpose of developing individualized activation engagement pathways post service or purchase. The process may involve securing and transporting personalized information. In some embodiments, the personalized information may be personal health information (PHI) and/or personal purchase information (PPI) from the point of purchase. The process may further involve delivery of the personalized information to the cloud. The process may further involve then using the personalized information to develop an individualized curriculum of engagement. The individualized curriculum of engagement may be delivered periodically to a suitable device in various digital formats. The digital formats include, but are not limited to, video, electronic mail (e-mail), text, picture, and regular paper mail. Where videos are used, videos are contextualized for the individual. A personalized web address, i.e., personalized uniform resource locator, PURL, is created which is then accessible to the individual on the various digital platforms. The digital platforms include but are not limited to the consumers' computers, mobile devices, and/or smart TVs.

In one embodiment, a method involves the implementation of the above-described process through the cloud environment. The method involves securely passing along the data from one system to another without compromising individual data set. This is performed using application programming interfaces (API). The method involves algorithms to dissever the information into actionable deliverables which are translated into a curriculum in the form of content. In one embodiment of the method, this content is then delivered to the consumer in the most appropriate form and time.

In some embodiments, the purchasing data are sourced from many areas. These areas may include, but are not limited to, monetary transactions at the point of sale. Such monetary transactions at the point of sale may have demographic and/or medical information, such as diagnosis, name of a purchased drug, form of purchase, amount paid during the purchase, contact information of the consumer (address, emails, phone number), historical purchases, and family purchases (sometimes tied to loyalty programs). The input data can also be sourced from other transactional areas, such as external devices like biometrics.

Another embodiment further seeks to address post-purchase engagement through integration of purchase data and personalized data in order to create a personalized engagement pathway. In this embodiment, the personalized engagement pathway is created to best fit the purchaser both in context, timing, and delivery, using, but not limited to, video media. The post-purchase engagement could incorporate historical personalized data, some of which could be distributed among various forums, current purchases or services offered, shared data sets, and scientific data sets to intelligently make the best decision on what pathway to create for the consumer. The decision on what pathway to create for the consumer may include the decision on how, how long, and which methodology is best to engage the consumer, i.e., engagement continuum.

In one embodiment, a method of transferring consumer data from the retail environment to personalized platforms is provided. The method includes securing, using at least one computer, individualized personalized information at a point of purchase. The method further includes transporting, using the at least one computer, the individualized information from the point to a webspace. The method further includes generating, using the at least one computer, individualized curriculum of engagement using the individualized information in the webspace. The method further includes delivering, using the at least one computer, the curriculum to a user.

Furthermore, provided in one embodiment is a method of transferring consumer data from the retail environment to personalized platforms, comprising: securing, using at least one computer, individualized personalized information at a point of purchase; transporting, using the at least one computer, the individualized information from the point to a webspace; generating, using the at least one computer, individualized curriculum of engagement using the individualized information in the webspace; and delivering, using the at least one computer, the curriculum to a user.

Furthermore, provided in one embodiment is a method of transferring user data from a point of interaction to an individualized curriculum of engagement, comprising: securing, using at least one computer, individualized personalized information at a point of interaction; transporting, using the at least one computer, the individualized information from the point of interaction to a webspace; generating, using the at least one computer, individualized curriculum of engagement using the individualized information in the webspace; and delivering, using the at least one computer, the curriculum to the user.

### Video Page Platform

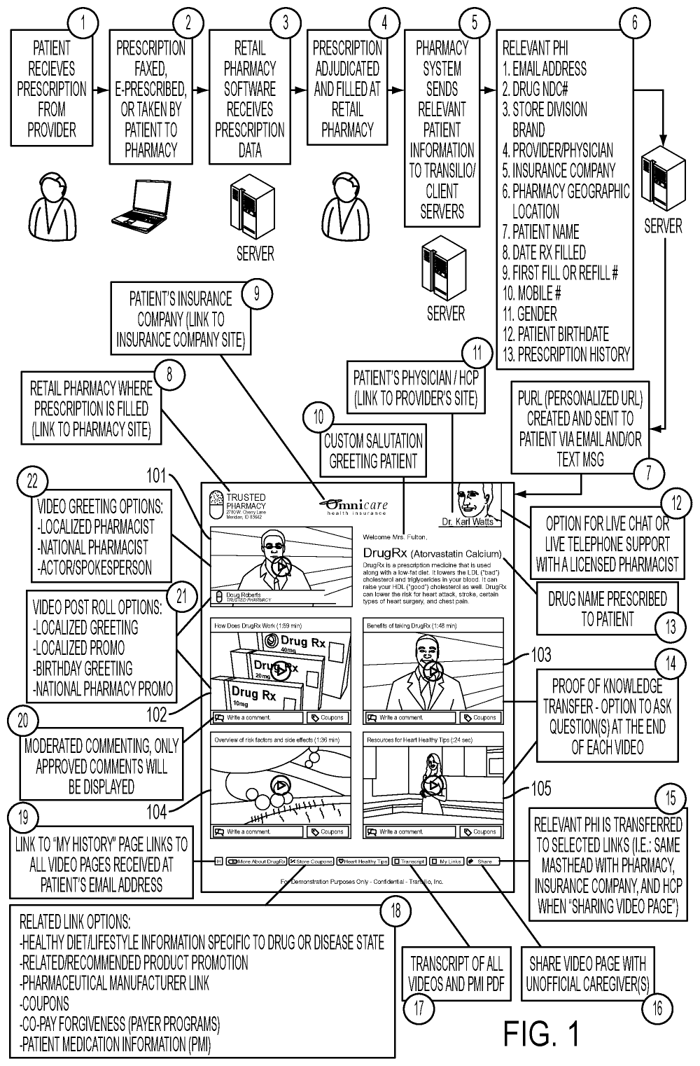

FIG. 1 is a schematic flow chart showing a personalized retail engagement pathway according to some embodiments.

At callout 1, a patient receives a prescription from a provider. The provider may be some health care provider that the patient has visited. At callout 2, the prescription is faxed, e-prescribed, or taken by the patient to the pharmacy. If faxed or e-prescribed, the provider or a party acting on behalf of the provider may be the party to send the prescription to the pharmacy. If the patient takes the prescription to the pharmacy, then it may be in the form of a written or printed prescription.

At callout 3, the retail pharmacy software receives the prescription data. The retail pharmacy software may receive the prescription data through various inputs. The prescription data may be received via an e-prescription, such as that sent by the provider. The prescription data may be received by manual input from an employee of the retail pharmacy. The prescription data may be received via other forms of input. At callout 4, the prescription is adjudicated and filled at the retail pharmacy.

At callout 5, the pharmacy system sends relevant patient information to a webspace. The webspace may be implemented using servers. At callout 6, various elements of information may be sent as the patient information, including: the patient's email address, the prescribed drug's NDC #, the store division brand, the provider/physician or an identifier thereof, the insurance company or an identifier thereof, the pharmacy's geographic location, the patient's name, the date the prescription was filled, an identifier of the number of fills such as the first fill or a refill count, the patient's mobile phone number, the patient's gender, and the patient's birthdate, the patient's prescription history. Other relevant information also may be sent.

At callout 7, a personalized URL is created in the webspace, such as by the servers. The personalized URL is sent to the patient. The personalized URL may be sent via email, text message, or some other communication mechanism.

Callouts 8 through 22 highlight various features of a communication delivered to the patient using the personalized URL. The information highlighted by callouts 8 through 22 may be information that is shown on a webpage, that webpage being loaded when the patient clicks or otherwise selects the personalized URL.

At callout 8, a logo, name, or some other branding or identifier of the retail pharmacy where the prescription was filled, is displayed. This information may include an address or other information for contacting the retail pharmacy. The information may act as a hyperlink to the pharmacy's website.

At callout 9, a logo, name, or some other branding or identifier of the patient's insurance company, is displayed. The information may act as a hyperlink to the insurance company's website.

At callout 10, a custom salutation greeting the patient is displayed. This may be generated based on the customer information received in the webspace, such as name, gender, etc.

At callout 11, a log, name, photo, or some other branding or identifier of the patient's physician or health care provider is displayed. The information may act as a hyperlink to the physician's or health care provider's website.

At callout 12, a hyperlink, phone number, or other information may be displayed to allow the patient to access a live chat or live telephone support line. This live chat or live telephone support feature may provide the patient access to a licensed pharmacist. This licensed pharmacist may be available in order to provide the patient more information about the prescribed drug or to answer questions from the patient about the prescribed drug.

At callout 13, the name of the drug prescribed to the patient is displayed. This may include a brand name as well as a drug name. As shown in this embodiment, a brand name “DrugRx” for the prescribed drug and a drug name “Atorvastatin Calcium” for the prescribed drug are displayed.

On the webpage, various videos are provided to the patient. Video 101 may provide a greeting to the patient. Video 102 may provide information on the basic function of the prescribed drug. As shown, video 102 is titled “How Does DrugRx Work.” Video 103 may provide information on the basic positive outcomes of the prescribed drug. As shown, video 103 is titled “Benefits of taking DrugRx.” Video 104 may provide information on possible negative outcomes of the prescribed drug. As shown, video 104 is titled “Overview of Risk Factors and side effects.” Video 105 may provide information as to other potentially useful health information for the patient. As shown, video 105 is titled “Resources for Heart Healthy Tips.” In other embodiments provided herein, a different number of videos, different content of the videos, a different arrangement of the videos, or other presentation of video content may be possible.

At callout 22, a video greeting is noted. The video greeting may be a greeting from a localized pharmacist, such as one selected based on the geographic location of the retail pharmacy where the prescription was filled. The video greeting may be from a national pharmacist. The video greeting may be from an actor or spokesperson.

At callout 21, various video post roll options are noted. A post roll may be a short video or other content shown after the end of another primary video. A localized greeting post roll video may be used. A localized promotional post roll video may be used. A birthday greeting post roll video may be used. A national pharmacy promotional post roll video may be used. Pre roll options that may be short videos or other content shown before the beginning of another primary video may also be used. Post roll and pre roll options may also include advertisements.

At callout 20, the various videos may include a comments section displayed or provided by hyperlink. The comments section may display comments provided by other patients who viewed or were otherwise provided with the same video. The comments may be moderated to enhance the quality and value of the comments displayed.

At callout 14, proof of knowledge transfer may be integrated with the videos. This feature may be used to verify that the patient understood the information provided in the videos. This feature may be provided as an option to ask or answer questions at the end of each video.

At callout 16, a “Share” link in provided so that the patient can share the video page with unofficial caregivers. Functionality may be provided so that the patient can specify what other party or parties should receive the shared video page.

At callout 17, a “Transcript” link is provided so that the patient can request and receive an electronic copy of the information presented on the video page in a text-based format. As such, the patient may be able to store the information presented in the videos and elsewhere on the video page in a format that the patient can later read without re-viewing the videos. This transcript may include relevant personalized information of the patient. When the patient clicks the “Transcript” link, the patient may be provided with a Portable Document Format (“PDF”) transcript that the patient can then save, print, or use in some other fashion.

At callout 19, a “My History” link is provided so that the patient can review other videos previously sent to the patient. The history feature may present the patient with a listing of video pages, including personalized URLs, previously delivered to the patient.

At callout 18, various other links may be provided on the video page. A “Store Coupons” link may be provided so that the patient can retrieve promotional offers and information. The promotional offers and information may include promotional offers and information in the form of coupons that the patient can redeem online, in the retail pharmacy, or in some other fashion. A link may be provided that gives the patient access to healthy diet and lifestyle information related to the prescribed drug or the condition that the prescribed drug is related to. A link may be provided that gives the patient access to promotional offers and information for products related to the prescribed drug or the condition that the prescribed drug is related to. A link may be provided that gives the patient access to recommended for a patient taking the prescribed drug or with the condition that the prescribed drug is related to. A link may be provided that gives the patient access to a website for the manufacturer of the prescribed drug. A link may be provided that gives the patient access to co-pay forgiveness (payer programs) that the patient may redeem with the insurance provider, physician, or some other party. A link may be provided that gives the patient access to patient medication information (“PMI”). A link may be provided that gives the patient access to more information about the prescribed drug. A link may be provided that gives the patient access to further general health information, such as “Heart Healthy Tips.” A link may be provided that gives the patient access to a list of recommend links, or “My Links,” for the patient.

At callout 15, it is noted that when a link is selected from the video page, relevant personalized information is transferred to the linked webpage. This relevant personalized information may depend on context. In some contexts, relevant personalize information may include the information presented about the retail pharmacy, insurance company, and health care provider, as described with respect to callouts 8, 9, and 11, respectively.

As shown in this figure, various points of information related to a retail or other point of engagement can be provided to the customer. The information is tailored to the customer's transaction at the point of engagement. One or more videos are utilized as an effective format for providing the information to the customer. Thereby, the embodiment shown in this figure provides effective post-purchase engagement with the customer by maintaining contact with the customer, providing relevant information, and allowing the customer to access at a future time other information that may improve the customer's experience with the product and encourage adherence to the product.

In the embodiment of FIG. 1, personalized information, five videos, and links were chosen to show an exemplary video page. In this instance, there is no restriction to be placed on other embodiments of the video page based on the exemplary selection of five videos engagements, the content of the five videos, the content of the links, or any other exemplary details of the figure.

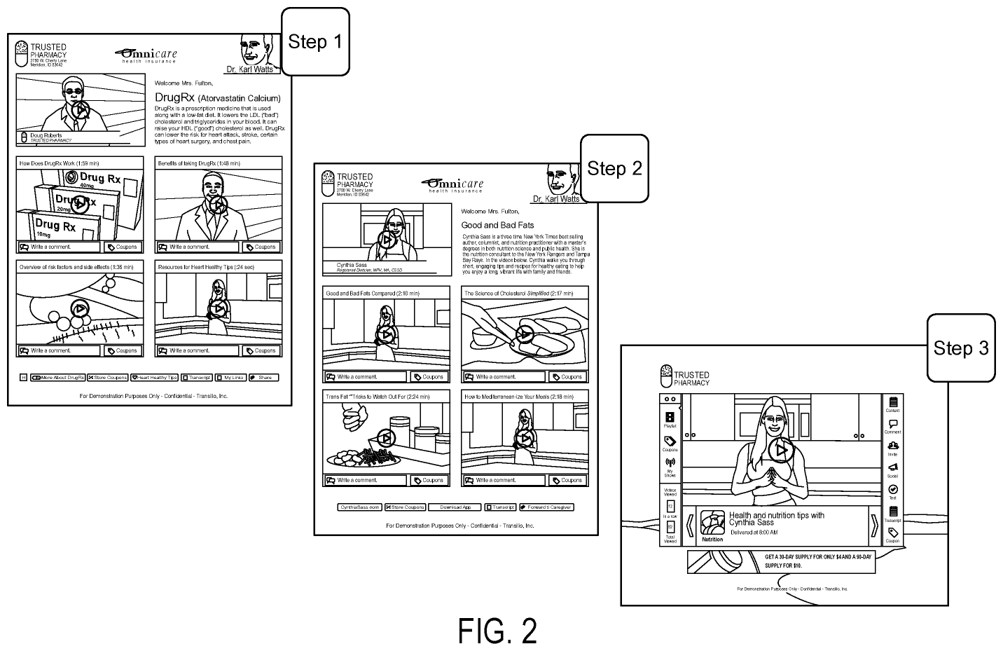

FIG. 2 is a diagram showing various steps of a personalized retail engagement pathway according to some embodiments.

The page displayed at the top-left of the figure and labeled “Step 1” may be a first video page in a personalized retail engagement pathway (“PREP”) for a patient. As shown in Step 1, a first video page may be delivered to the patient after the patient has received a prescription drug. This video page may contain information targeted particularly to the prescription drug that the patient has just received, and more generally to the condition that the patient may be receiving treatment for. In some embodiments, the video page described with respect to FIG. 1 may be used as the first page delivered to the patient in Step 1.

The page displayed at the center of the figure and labeled “Step 2” may be a second video page in a personalized retail engagement pathway for a patient. As shown in Step 2, a second video page may be delivered to the patient after the patient has received a prescription drug and after the patient has received the first video page. The delivery of the second video page of Step 2 may be done at a time selected based on a schedule for the personalized retail engagement pathway. This time may be selected so as to engage the patient in an ongoing fashion. As further shown, the second video page may contain information, including videos, that is different from the information presented in the first video page. The content of the second video page may be selected as content that is more relevant at the time the second video page is delivered, such as based on an expected pattern of usage of the prescription drug by the patient. As shown in the video page of Step 2, a page concerning “Good and Bad Fats” may be provided. This video page may contain a video with a greeting from a subject matter expert, such as the exemplary Cynthia Sass, a registered dietician. The video page may contain a video with information as to “Good and Bad Fats Compared,” “The Science of Cholesterol Simplified,” “Trans Fat ‘Tricks’ to Watch Out For,” and “How to Mediterranean-ize Your Meals.” The video page of Step 2 may contain similar information as to the retail pharmacy, insurance company, health care provider, and links as described with respect to the video page of FIG. 1.

The page displayed at the bottom-right of the figure and labeled “Step 3” may be a third video engagement in a personalized retail engagement pathway for a patient. As shown in Step 3, a single video that is not a complete video page may be delivered to the patient after the patient has received a prescription drug, after the patient has received the first video page, and after the patient has received the second video page. The delivery of the third video engagement of Step 3 may be done at a time selected based on a schedule for the personalized retail engagement pathway. This time may be selected so as to engage the patient in an ongoing fashion. As shown, the third video engagement may be delivered to the patient using a standalone and/or web-enabled software application that serves as a video delivery application for the client. The software delivery application may contain features as described with respect to the daily push platform described later in this disclosure. As further shown, the video engagement may contain information, including video that is different from the information presented in the first video page and the second video page. The content of the third video engagement may be selected as content that is more relevant at the time the third video engagement is delivered, such as based on an expected pattern of usage of the prescription drug by the patient.

As shown in FIG. 1, a personalized retail engagement pathway leverages the effective post-purchase engagement features of the video page of FIG. 1 in a planned series of engagements that encourage long term engagement with the patient. Thereby, the embodiment shown in this figure provides effective post-purchase engagement with the customer by maintaining contact with the customer more than a single point after the transaction, providing relevant information on an ongoing basis, and allowing the customer to continue to access at a future time other information that may improve the customer's experience with the product and encourage adherence to the product.

In the embodiment of FIG. 2, the first, second, and third video engagements were chosen to be an exemplary personalized retail engagement pathway. There is no restriction to be placed on other embodiments of the personalized retail engagement pathway based on the exemplary selection of three video engagements, the content of the three video engagements, or any other exemplary details of the figure.

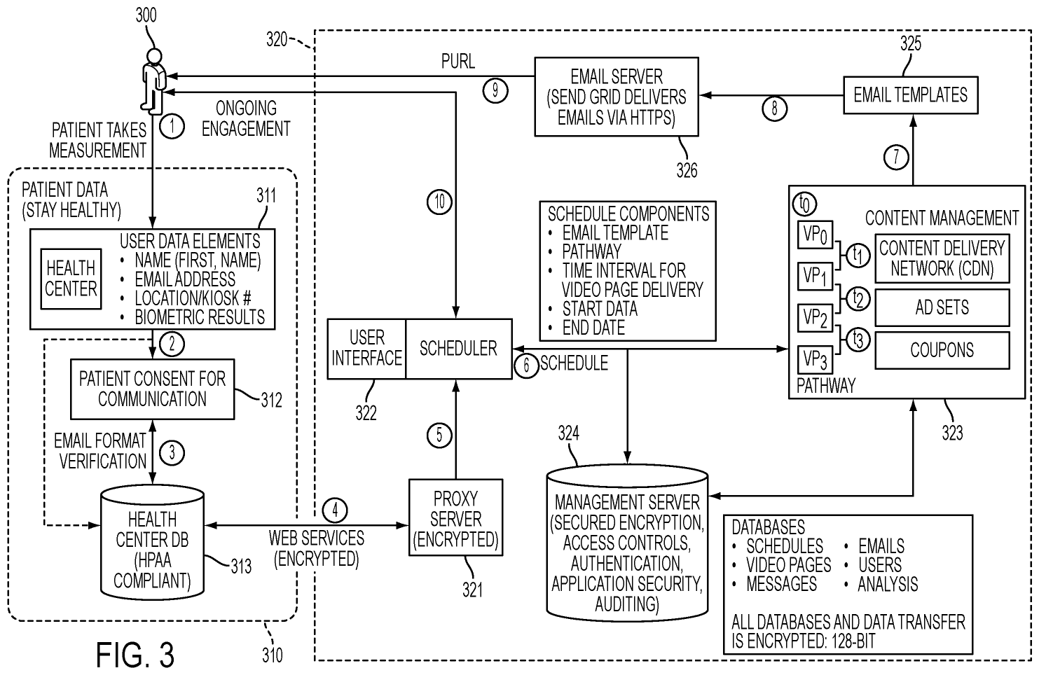

FIG. 3 is a system diagram showing elements in a system for user engagement according to some embodiments. Various steps in a process using the system are shown.

A user 300 is a patient and a target for engagement by the system. At step 1, the user 300 interacts with a health center kiosk 311 of the patient data environment 310. The user 300 takes measurements and enters them. The user 300 may enter other information as well. The information entered at health center kiosk 311 may include: the patient name, the patient's email address, the location of the kiosk, the kiosk number, and biometric results. At step 2, the user 300 interacts with patient consent for communication element 312. The user provides consent for the sharing of the user information as necessary, as well as for the receipt of communications from the system. At step 3, the user's email address format is verified. The user information is stored in the health center database 313.

The system contains a secured environment 320, which in turn contains a proxy server 321, a scheduler component 322, a content management component 323, a management server 324, email templates component 325, and an email server 326.

At step 4, the proxy server 321 communicates with the health center database 313 using secure web services. User information is retrieved as necessary for the other components of secured environment 320. At step 5, proxy server 5 communicates with scheduler component 322. The proxy server 321 provides user information to scheduler component 322 in order to schedule engagements with the user. Scheduler component 322 may include a user interface component in order to receive schedule information input from the user 300 or another user.

At step 6, the scheduler component 322, the content management component 323, and the management server 324 communicate. These components communicate in order to prepare a personalized engagement pathway for the user 300. The schedule may entail information including: an email template, a pathway, a time interval for delivery of video pages, a start date, and an end date. The management server 324 may perform functions related to access controls, authentication, application security, and auditing. The management server may retain information related to schedules, video pages, messages, emails, and analytics. The content management component may have access to a content delivery network, ad sets, and coupons. The personalized engagement pathway for the user 300 may include four video pages: VP0, VP1, VP2, and VP3. The delivery of the video pages may be scheduled to start at time t0, with time delays of t1, t2, and t3 before delivery of the next VP1, VP2, and VP3, respectively.

At step 7, the content management component 323 communicates with email templates component 325 in order to generate an email to the user 300 for a scheduled engagement. At step 8, email templates component 325 communicates with email server 326 in order to prepare an email for delivery to user 300. At step 9, email server 326 sends an email to user 300 included a personalized URL for the scheduled video page. The email may be sent via a secured transmission, such as via HTTPS.

At step 10, ongoing engagements take place between the system and user 300.

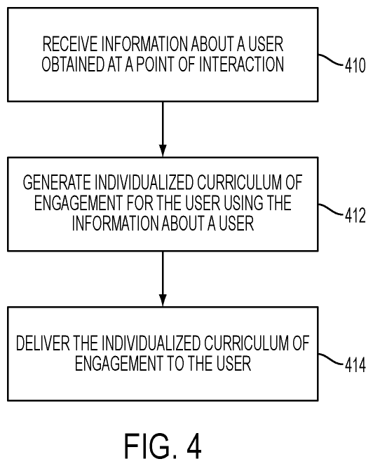

FIG. 4 is a flowchart showing a process for user engagement according to some embodiments. At step 410, information about a user obtained at a point of interaction is received. At step 412, an individualized curriculum of engagement is generated for the user using the information about the user. At step 414, the individualized curriculum of engagement is delivered to the user.

An individualized curriculum of engagement is a planned series of engagements and accompanying information relevant to the user, wherein the information is tailored to the particular user based on the user information received. An individualized curriculum of engagement may include video and/or other forms of information, such as those discussed previously in this disclosure. An individualized curriculum of engagement may include a personalized engagement pathway, such as a personalized retail engagement pathway. An individualized curriculum of engagement may include video pages, such as video pages incorporated into a personalized engagement pathway.

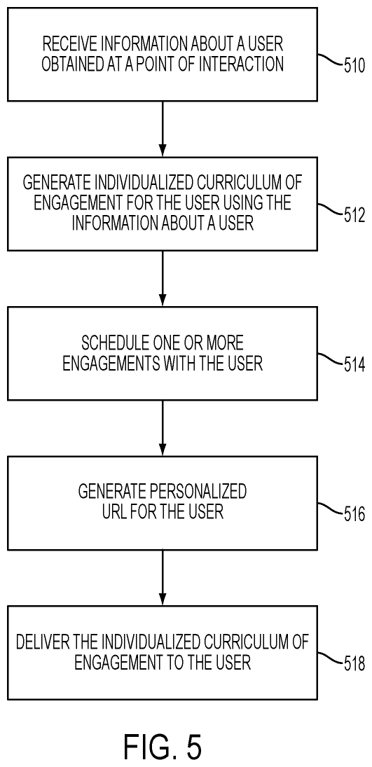

FIG. 5 is a flowchart showing a process for user engagement according to some embodiments. At step 510, information about a user obtained at a point of interaction is received. At step 512, an individualized curriculum of engagement is generated for the user using the information about the user. At step 514, one or more engagements are scheduled with the user. At step 516, a personalized URL is generated for the user. At step 518, the individualized curriculum of engagement is delivered to the user.

In some embodiments, delivering the individualized curriculum of engagements in step 518 may be accomplished by sending the personalized URL to the user, such as via email or text message. In some embodiments, the actions of steps 514 and 516 may be part of generating an individualized curriculum in step 512. In such cases, the scheduled engagements may constitute the series of engagements that make up a personalized engagement pathway as part of the individualized curriculum of engagement. In some embodiments, these scheduled engagements may be video engagements that constitute a personalized engagement pathway as part of the individualized curriculum of engagement. In some embodiments, various steps may be repeated, such as repeating steps 516 and 518 when more than one engagement is scheduled with the user.

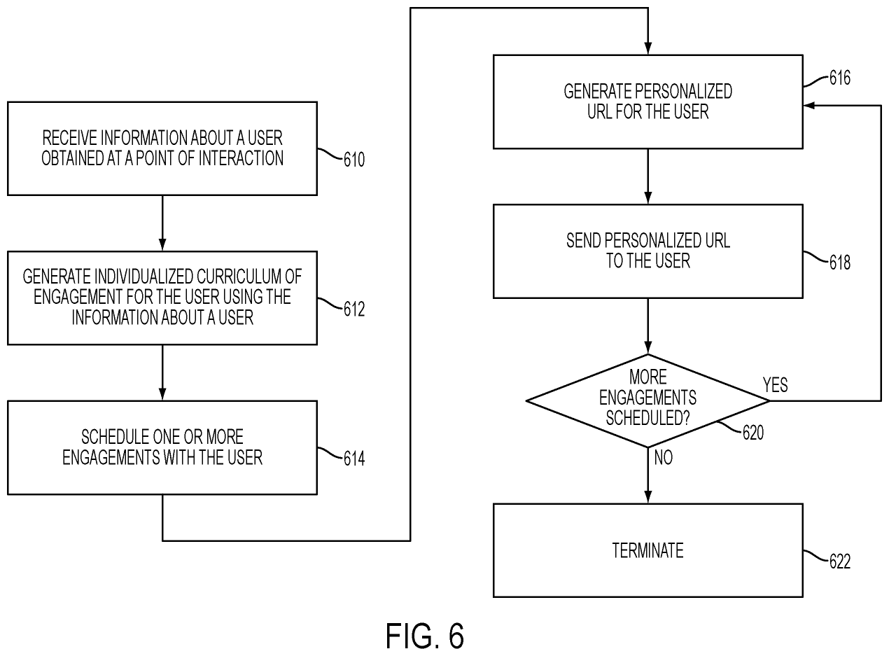

FIG. 6 is a flowchart showing a process for user engagement according to some embodiments. At step 610, information about a user obtained at a point of interaction is received. At step 612, an individualized curriculum of engagement is generated for the user using the information about the user. At step 614, one or more engagements are scheduled with the user. At step 616, a personalized URL is generated for the user. At step 618, the personalized URL is sent to the user. At step 620, a determination is made as to whether more engagements are scheduled. If more engagements are scheduled, then the process continues at step 616. If no more engagements are scheduled, then the process terminates at step 622.

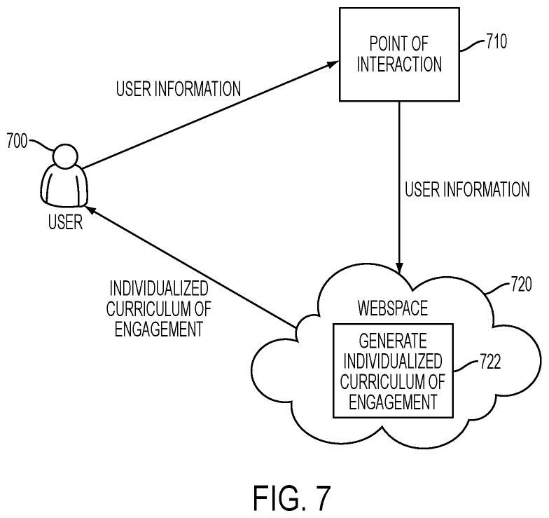

FIG. 7 is a diagram showing elements in a system and movement of information in a process for user engagement according to some embodiments. A user 700, a point of interaction 710, and a webspace 720 are provided. Thus user 700 may be a consumer of some product or service related to point of interaction 710. Based on some interaction or transaction related to the product or service, user information is gathered at the point of interaction 710. This information may be gathered by a computing device provided at the point of interaction. The user information is then sent to the webspace 720. The webspace 720 may be a computing device, a server, numerous servers, or any other network-connected computing platform. In the webspace 720, a step 722 of generating an individualized curriculum of engagement takes place using the user information. The webspace then delivers the individualized curriculum of engagement to the user 700. The generation of the individualized curriculum of engagement may include generating a personalized engagement pathway. The delivery of the individualized curriculum of engagement may include delivering personalized URLs for various elements of the personal engagement pathway, such as sending a personalized URL to the user 700 each time a video page is scheduled to be delivered to the user 700 as part of the personalized engagement pathway. The delivery may be performed by delivering an email, text message, or other communication to a user computer device, such as a smartphone, cellular phone, tablet computer, laptop computer, or desktop computer.

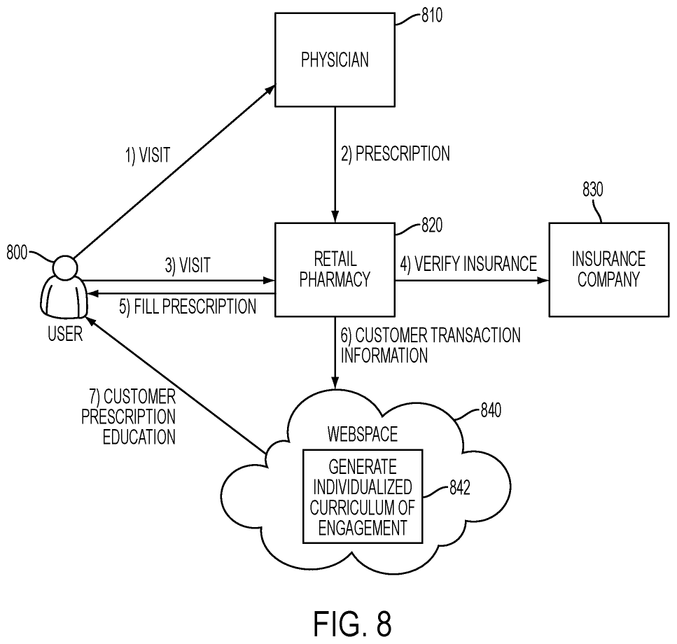

FIG. 8 is a diagram showing elements in a system and movement of information in a process for user engagement according to some embodiments. A user 800, a physician 810, a retail pharmacy 820, an insurance company 830, and a webspace 840 are involved in this embodiment. Thus user 800 may be a patient, wherein insurance company 830 is an insurance provider to patient 800. At step 1, the user 800 visits the physician 810. At step 2, prescription information is sent from the physician 810 to the retail pharmacy 820. The prescription information may correspond to a prescription prescribed by the physician to the user. At step 3, the user 800 visits the retail pharmacy 820. At step 4, the retail pharmacy 820 verifies the insurance of the user 800 with the insurance company 830. At step 5, the retail pharmacy 820 fills the prescription for the user 800. At step 6, the retail pharmacy 820 sends customer transaction information to the webspace 840. The customer transaction information may correspond to personalized information about the user 800, the prescription, the insurance company 830, the physician 810, or other relevant information, such as previously described in this disclosure. In webspace 840, a step 842 of generating an individualized curriculum of engagement takes place using the user information. At step 7, customer prescription education is delivered from webspace 840 to user 800.

In some embodiments, the various elements, steps, and information of this figure may correspond to features described previously in this disclosure. The physician 810 and/or the retail pharmacy 820 may be considered the point of interaction previously described. The customer transaction information may be the user information, personalized information, personalized health information, or other information about a user or consumer described previously in this disclosure. The customer prescription education may be an individualized curriculum of engagement, a personalized engagement pathway, one or more video pages, or any other engagement communication described previously in this disclosure.

### Additional Details

The availability of video content on the internet continues to increase at a rapid pace. Often with the increase in volume of video content comes an overwhelming amount of information and a decrease in the confidence that the information is reliable. The typical user today wants access to content streamed via multiple platforms such as web browsers and applications on mobile devices including smart phones and tablets. Additionally, there are vast amounts of information that companies are trying to communicate on a wide array of topics for both internal audiences as well as external audiences. The written word will only be remembered by a percentage of people.

The old phrase “a picture is worth a thousand words” may take on a whole new meaning when the messages are delivered via video. The ability to effectively communicate information with greater success through video is integral to the successful retention by the audience. The embodiments provided herein include a web and mobile client for consuming video content and a web administration tool for configuration and adding content. The embodiments provided herein have the ability to deliver trusted short form video content the end user needs and allow the user to return to the content as needed.

In some embodiments, HTMLS and CSS can be utilized for the presentation layer, ASP.NET MVC for the application logic, and MS-SQL for the storage of data, all running on Windows OS. Integration with a third party content delivery network (“CDN”) can be used to deliver the live video stream to end users. ASP.NET Version, MVC Framework, MS-SQL Version and configuration (SQL Azure), Hosting environment, CON partner. Deployments, management, user access, content uploads, CDN interaction.

In some embodiments, the purpose of a video page platform is to deliver content to users that is centered on rich media.

In some embodiments, a web client is provided. Online consumption of content by end users through a web client may include:
User Authentication (who can access the system
   Integrated with Social Networks—Google, Yahoo, Facebook, MSN Live
Content Delivery
   Header
      Personalized page containing relevant information
         Sponsor info
         Logo, Sponsor Name, Address, Phone #
         i.e. Department, Pharmacy, Organization Branch
         Referrer info
         Logo, Referrer Name, Address, Phone #
         i.e. Company, Healthcare provider, Grocery chain
      Body
         Greeting video
         i.e. Geo-local, National pharmacist, Actor/Spokespers
         Body Video(s)
         One or more videos related to subject
         i.e. Risk factors, side effects, resources
         Post-Roll
         i.e. Geo-local greeting, geo-local promo, national pharmacy promo, birthday greeting
         Subject name
         Subject description
      Commenting
         Other users able to comment on specific video
         Curated—only approved comments will be displayed
      Footer
         History
         Listing of all prior pages assigned
         i.e. related product info, coupons, Patient Medication Information (PMI)
         Transcripts of video
         Share
         Email
         Twitter
         Facebook
Content consumption (tracking user's consumption)
   Anonymous user consumption
      Content discovery/Browsing/preview (within Org)
   Registered user consumption
      System delivers based on subscription and/or assignment
      Content discovery I Browsing/preview (within Org)
Content subscription
   Per Org's organization of content
      Categories, groups, topics
User Profile
   Including First Last Name, User
   Minimal implementation
   Not required to view Content
   Name and password edit
Mobile Friendly

In some embodiments, a mobile client is provided. Online consumption of content by end users through a mobile client may include:
User Authentication (who can access the system)
   Integrated with Social Networks—Google, Yahoo, Facebook, MSN Live
Content Delivery
   Header
      Personalized page containing relevant information
         Sponsor info
         Logo, Sponsor Name, Address, Phone #
         i.e. Department, Pharmacy, Organization Branch
         Referrer info
         Logo, Referrer Name, Address, Phone #
         i.e. Company, Healthcare provider, Grocery chain
   Body
      Greeting video
         i.e. Geo-local, National pharmacist, Actor/Spokesperson
      Body Video(s)
         One or more videos related to subject
         i.e. Risk factors, side effects, resources
         Post-Roll
         i.e. Geo-local greeting, geo-local promo, national pharmacy promo, birthday greeting
      Subject name
      Subject description
   Commenting
      Other users able to comment on specific video
      Curated—only approved comments will be displayed
   Footer
      History
         Listing of all prior pages assigned
      i.e. related product info, coupons, Patient Medication Information (PMI)
      Transcripts of video
      Share
         Email
         Text
         Twitter
         Facebook
Content consumption
   Registered user consumption
      System delivers based on subscription and/or assignment
      Content discovery I Browsing/preview {within Org)
Content subscription
   Per Org's organization of content
      Categories, groups, topics
User Profile
   Including First Last Name, User
      Not required to view Content
      Name and password edit

In some embodiments, a web administration interface is provided for platform management, content administration, etc. Consumption of content by users through such an interface may include:
Organization Management {Super User Role—Transilio only at launch)
   Creation, editing, disabling of organizations
User Management
   Creation, editing, disabling of users
Content Management
   Creation, editing, disabling of content by organizations and platform admins
      Content collections
      Media elements {i.e. Videos, PDFs, etc.)
Permissions {media level, view/manage)
   Phase 1 default to Public
Analytics
   Reports on Total Users/Org, Total Pages and Videos viewed/date range

In some embodiments, a web services interface is provided. Consumption of content through such an interface may include:
Client services
   User Authentication
   Content Consumption
   Content Subscription
   User Profile
   Sharing
   Notifications

In some embodiments, there may be multiplatform content distribution (mobile and web) including:
Integration with Content Management System provider (i.e. Ooyala) for content browsing
Org User will interact with provider directly for media management

In some embodiments, the system may be scalable (mobile and web) including:
User Population of 50,000 concurrent users
Page Load Times of 3-5 seconds maximum

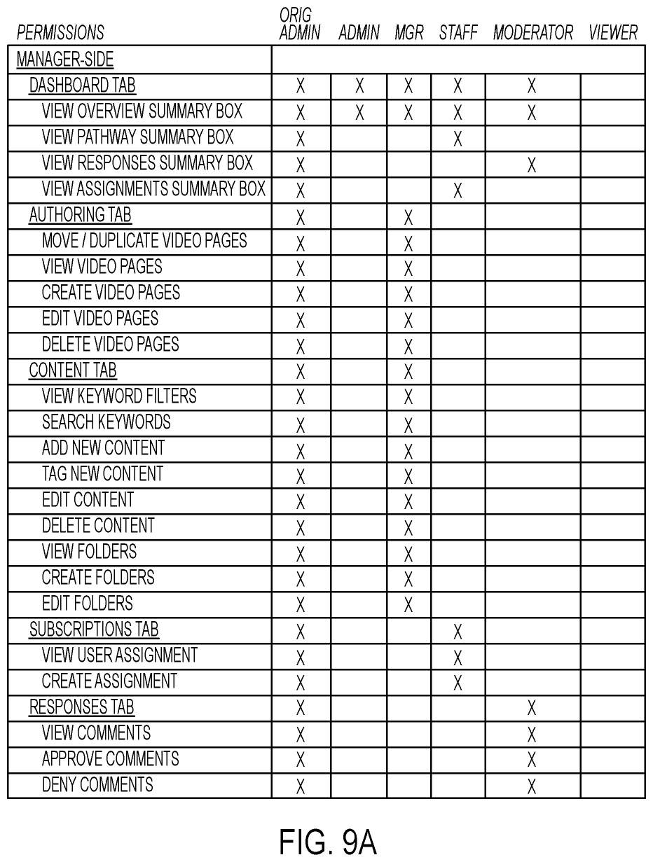

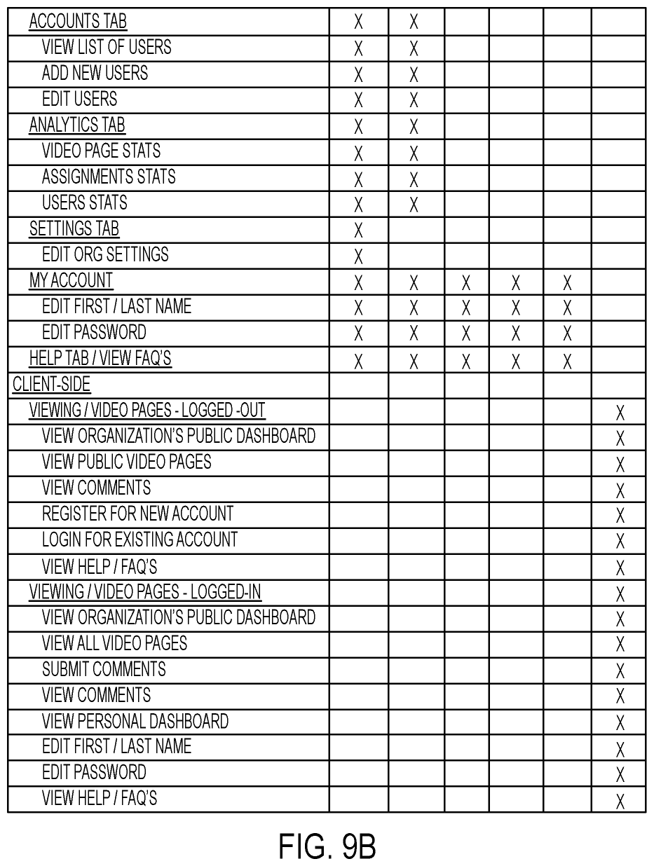

FIGS. 9A and 9B together show a roles/permissions matrix according to some embodiments. As shown, various permissions are mapped to various roles of users in the system. An “X” indicates that a user of that role has permission to perform the indicated function.

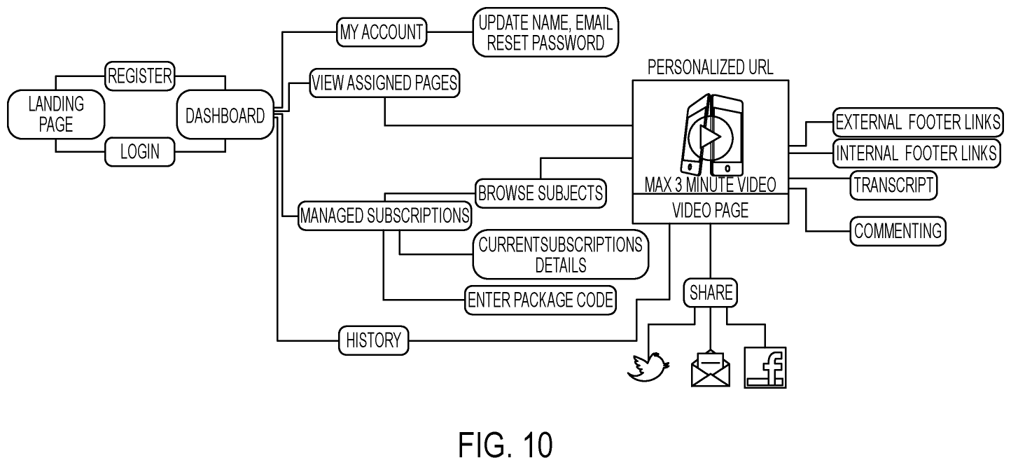

FIG. 10 shows a client site workflow according to some embodiments. As shown, various pages allow a user accessing the client interface to navigate to various other pages and perform various functions.

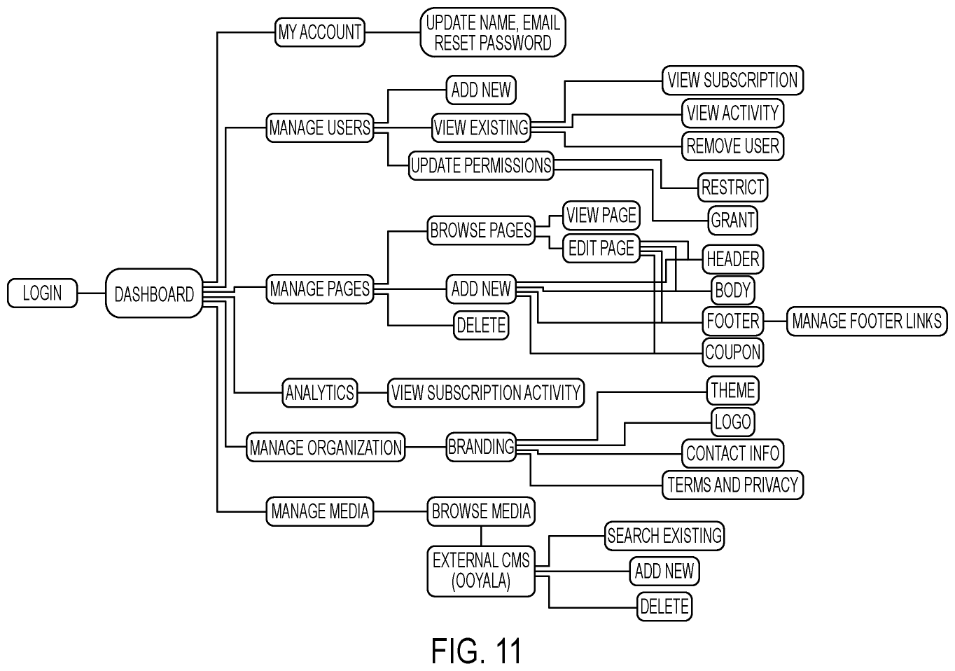

FIG. 11 shows an admin site workflow according to some embodiments. As shown, various pages allow a user accessing the admin interface to navigate to various other pages and perform various functions.

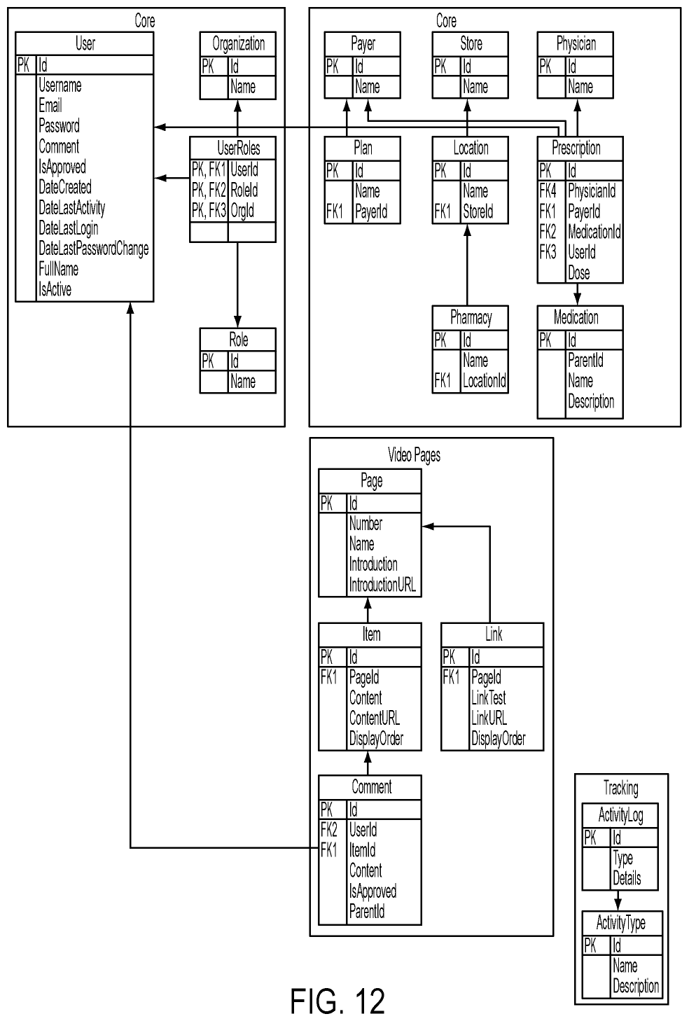

FIG. 12 shows a data model according to some embodiments. As shown, various data elements are organized into tables of a relational database model.

### Daily Push Platform

According to some embodiments, the purpose of a push platform system is to deliver push content that is centered on rich media. The system may push content to consumers on a scheduled basis.

In some embodiments, a web client is provided. Online consumption of content by end users through a web client may include:
User Authentication (who can access the system)
   Integrated with Social Networks—Google, Yahoo, Facebook, MSN live
Content consumption (tracking user's consumption)
   Anonymous user consumption data
   Package codes
   Content discovery/Browsing/preview (within channel)
Content subscription (channels, programs, episodes)
Commenting
User Profile
   Including First Last Name, User
   Minimal implementation□
   Not required to view Content□
   Name and password edit
Mobile Friendly
Sharing
   Email
   Social site integration□
Web browser push notifications (messages about content scheduling)

In some embodiments, a mobile client is provided. Online consumption of content by end users through a mobile client may include:
User Authentication (who can access the system)
   Integrated with Social Networks—Google, Yahoo, Facebook, MSN Live
Content consumption (tracking user's consumption)
   Anonymous user consumption data
   Content discovery/Browsing/preview (within channel)
Content subscription (channels, programs, episodes)
Commenting
User Profile
   Including First Last Name, User
   Minimal implementation
   Not required to view Content
   Name and password edit
Sharing
   Email
   Text
   Social site integration
Native mobile push notifications (messages about content scheduling)

In some embodiments, a web administration interface is provided for platform management, content administration, etc. Consumption of content by users through such an interface may include:
Organization Management (Super User Role—Transilio only at launch)
   Creation, editing, disabling of organizations
User Management
   Creation, editing, disabling of users
   Invitations
   Subscriptions
   Editing user subscriptions
Content Management
   Creation, editing, disabling of content by organizations and platform admins
      Content collections (Channels, Shows, Episodes, etc.)
      Media elements (i.e. Videos, PDFs, etc.)
Permissions (media level, view/manage)
   Analytics
   Total users/Subscriptions, Total Episodes viewed/date range

In some embodiments, a web services interface is provided. Consumption of content through such an interface may include:
Client services
User Authentication
Content Consumption
Content Subscription
User Profile
Sharing
Notifications

In some embodiments, there may be multiplatform content distribution (mobile and web) including:
Integration with Content Management System provider (i.e. Ooyala) for content browsing
Org User will interact with provider directly for media management

In some embodiments, the system may be scalable (mobile and web) including:
User Population of 50,000 concurrent users
User Population of 50,000,000 registered users
Page Load Times of 3-5 seconds maximum

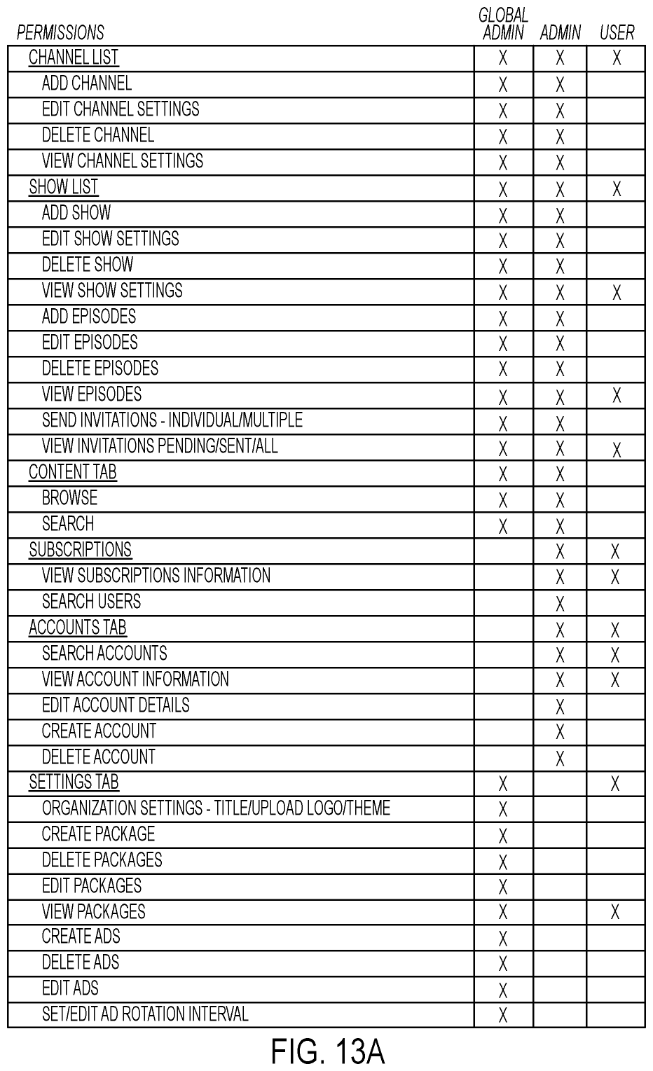

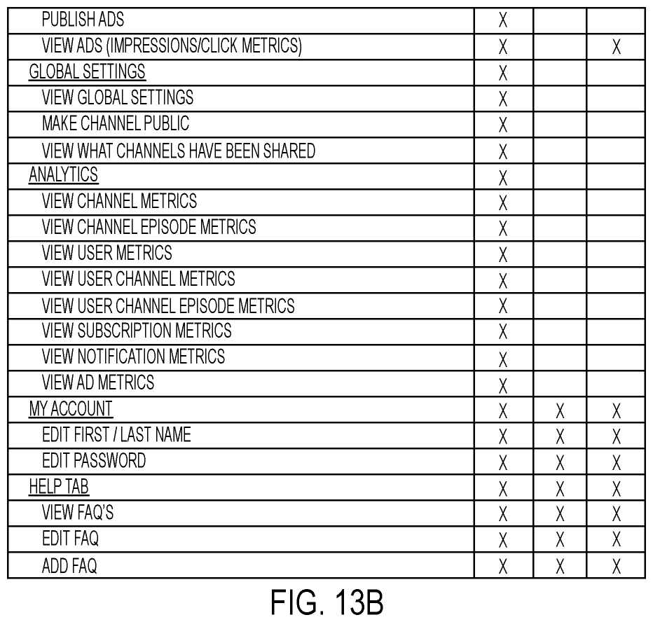

FIGS. 13A and 13B together show a roles/permissions matrix according to some embodiments. As shown, various permissions are mapped to various roles of users in the system. An “X” indicates that a user of that role has permission to perform the indicated function.

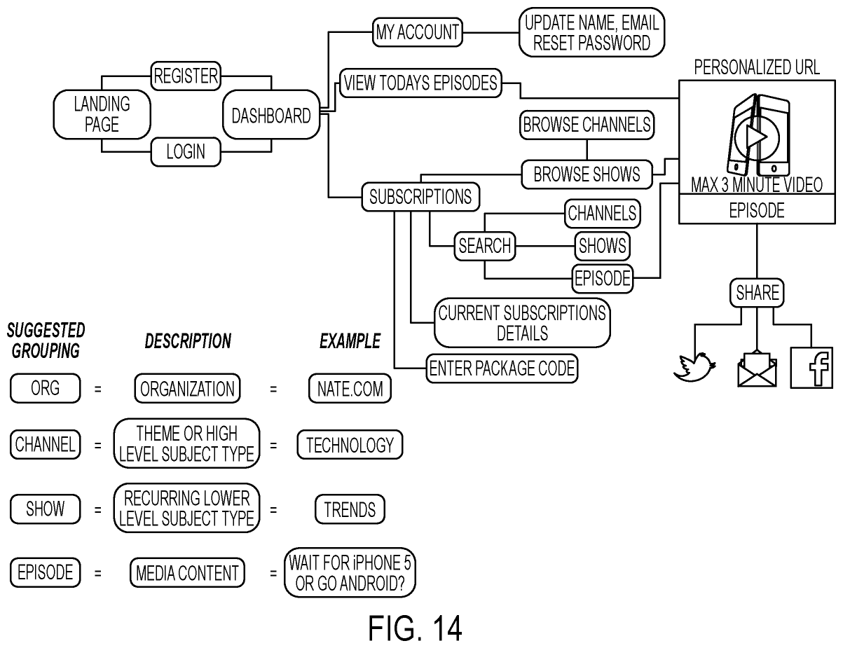

FIG. 14 shows a client site workflow according to some embodiments. As shown, various pages allow a user accessing the client interface to navigate to various other pages and perform various functions.

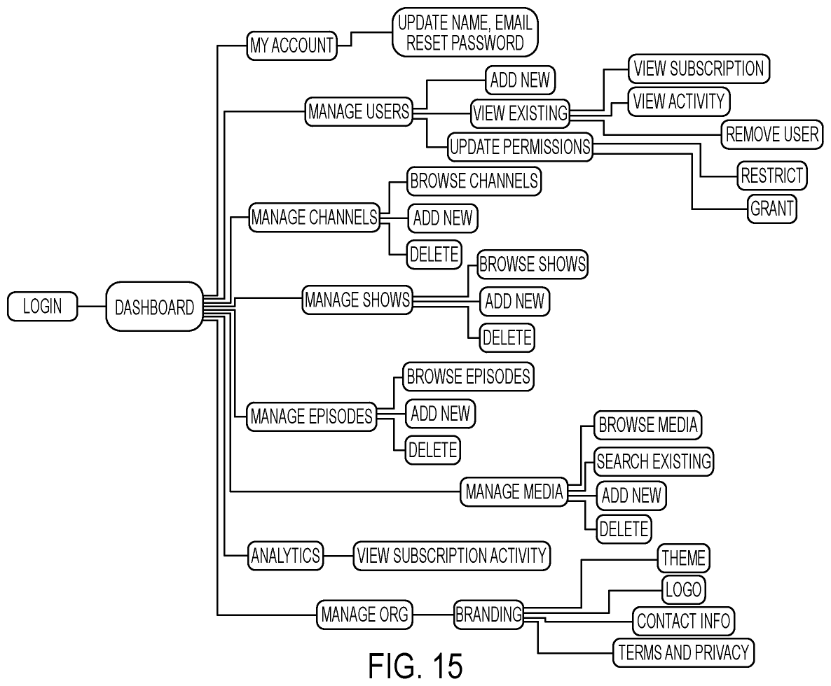

FIG. 15 shows an admin site workflow according to some embodiments. As shown, various pages allow a user accessing the admin interface to navigate to various other pages and perform various functions.

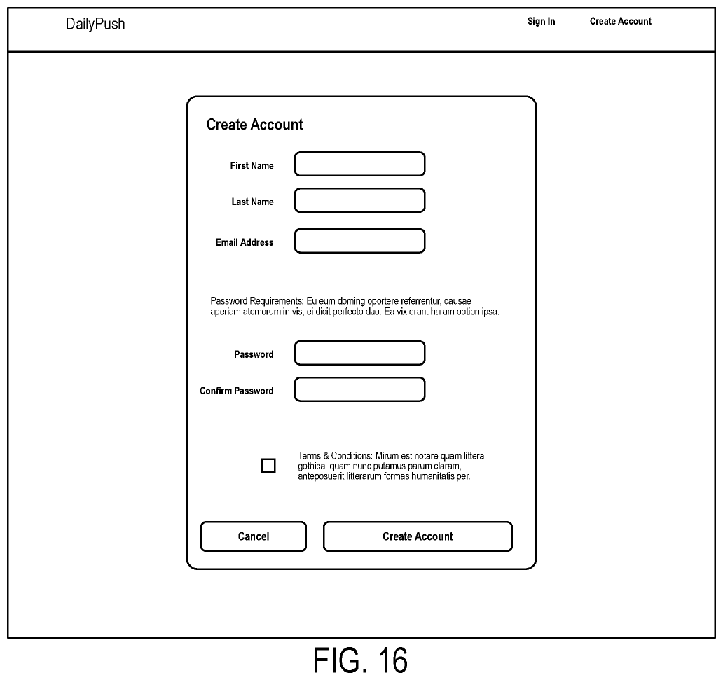

FIG. 16 shows an exemplary account creation interface according to some embodiments. As shown, a user can create an account to access the additional features of the system as well as maintain personal preferences.

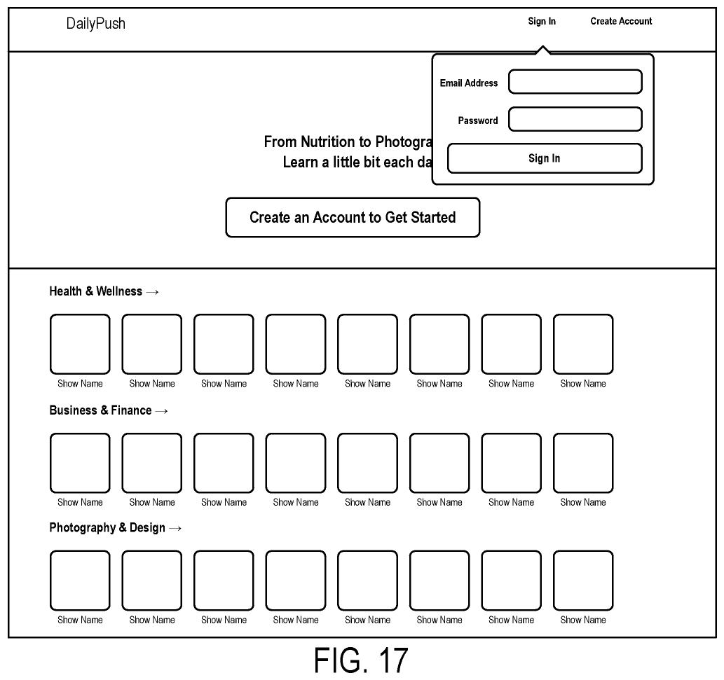

FIG. 17 shows an exemplary front end sign in interface according to some embodiments. As shown, a user on a page displaying video content can click a “Sign In” link and enter an email address and password to sign in.

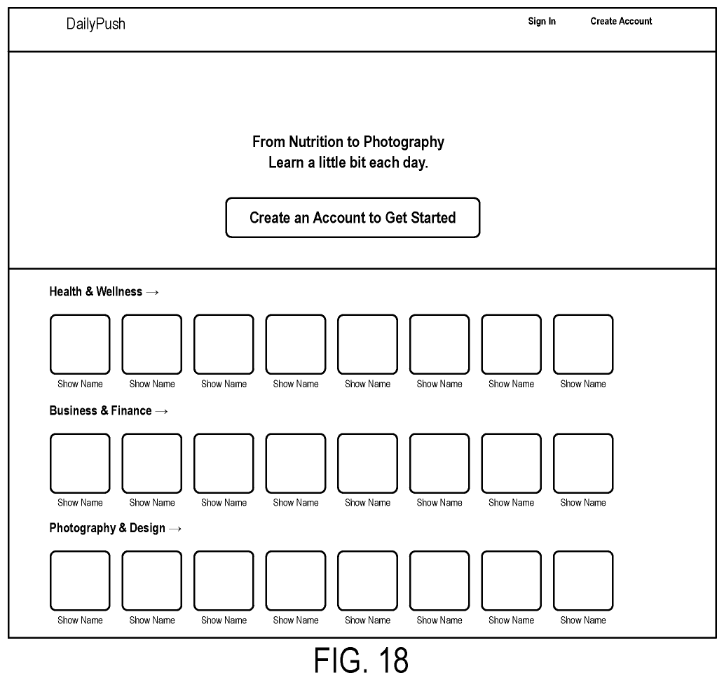

FIG. 18 shows an exemplary front end landing page interface according to some embodiments. As shown, a user may arrive on this webpage as a first page upon visiting the web site.

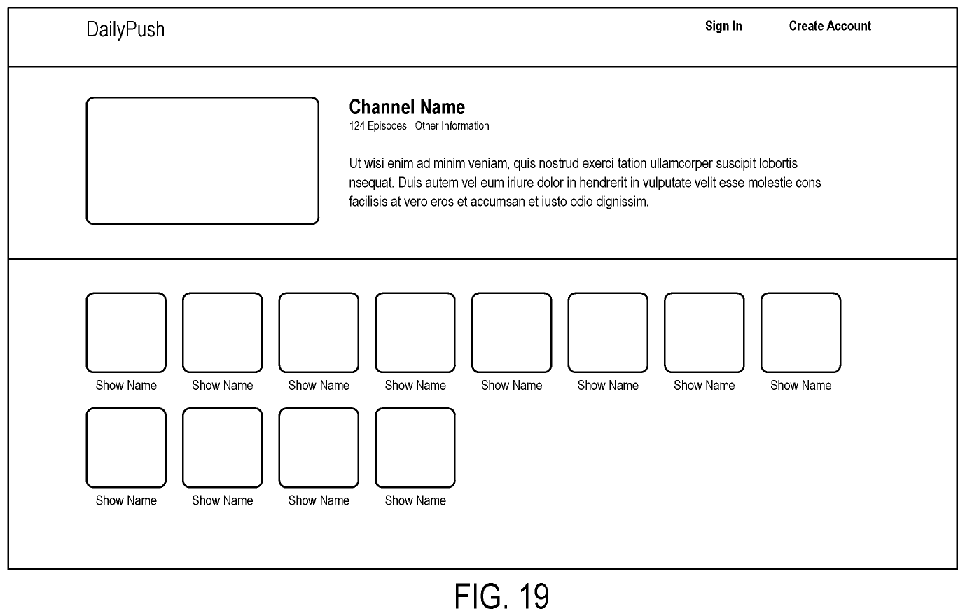

FIG. 19 shows an exemplary channel home page interface according to some embodiments. As shown, a user may view information about a channel of videos, including some or all of the videos associated with that channel.

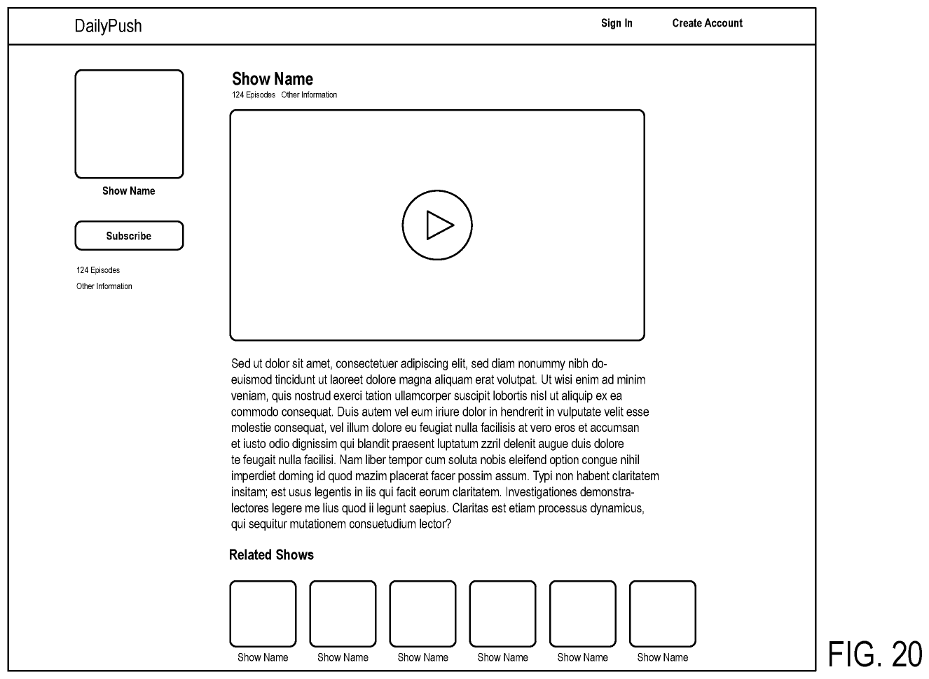

FIG. 20 shows an exemplary show home page interface according to some embodiments. As shown, a user may view information about a show, including a video associated with the show, a description of the show, and links to related videos.

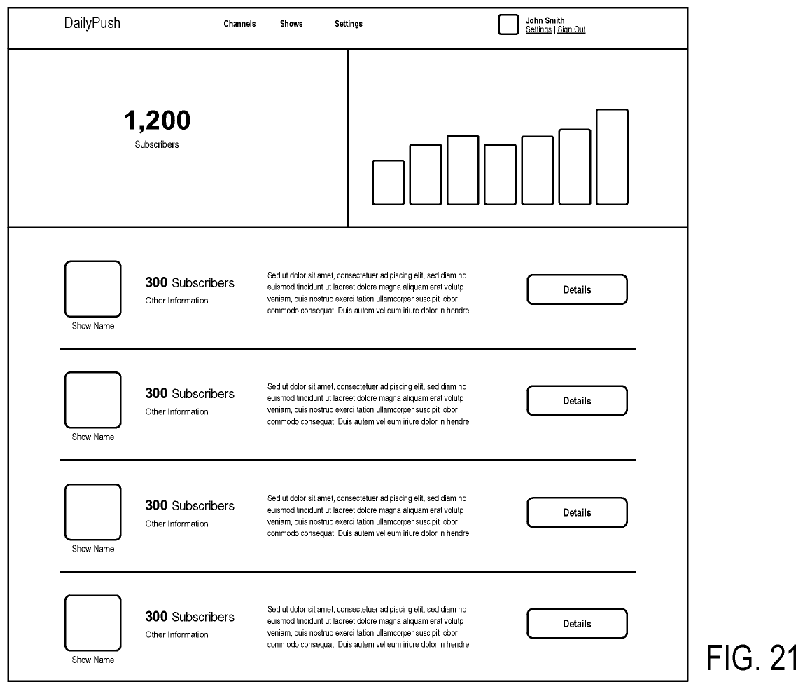

FIG. 21 shows an exemplary admin dashboard interface according to some embodiments. As shown, a user may view information channels, shows, subscribers, and other information relevant to that admin user.

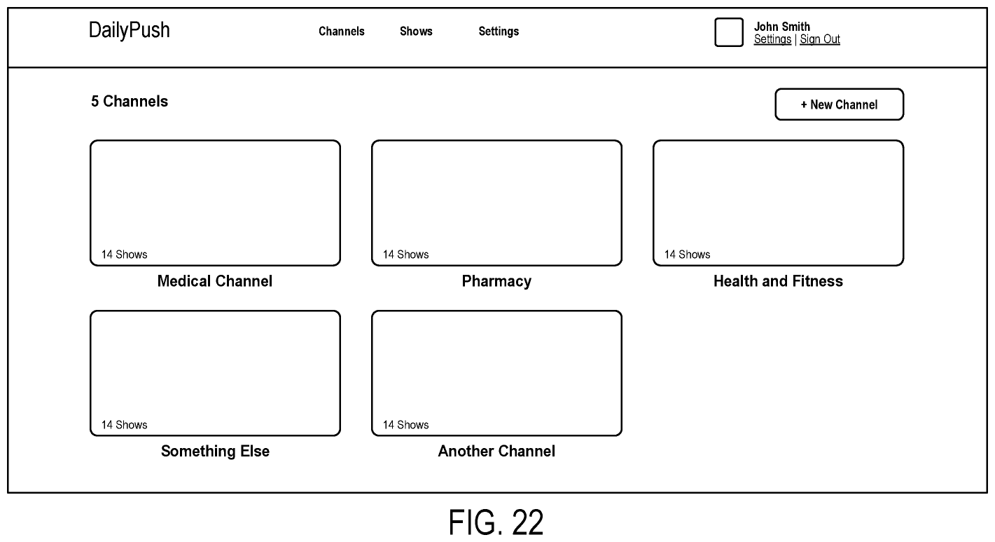

FIG. 22 shows an exemplary channel list interface according to some embodiments. As shown, a user may view a list of channels relevant to that admin user and add a new channel.

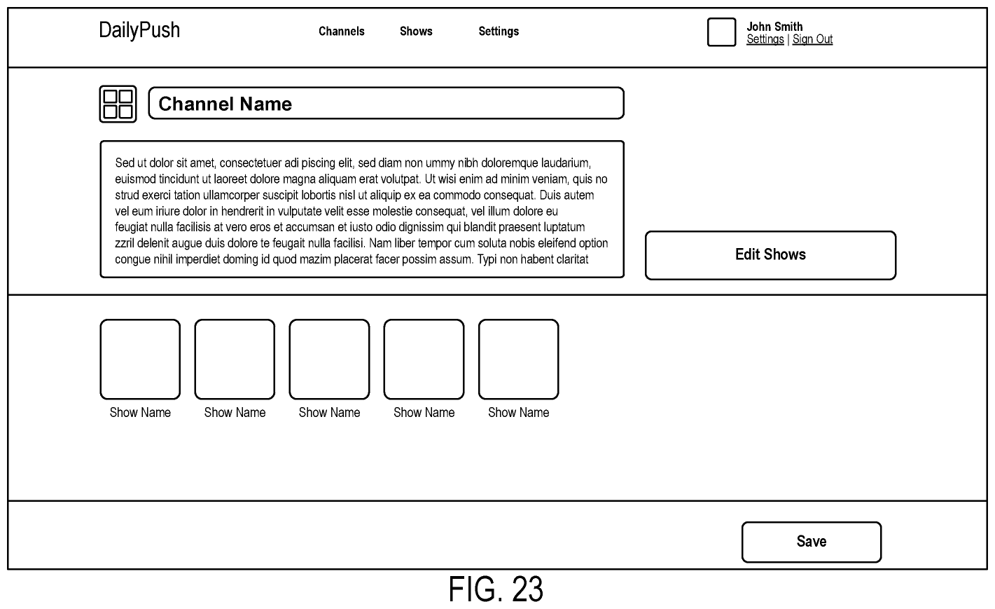

FIG. 23 shows an exemplary channel maintenance interface according to some embodiments. As shown, a user may view edit information about the channel, such as a name, a description, and videos associated with the channel.

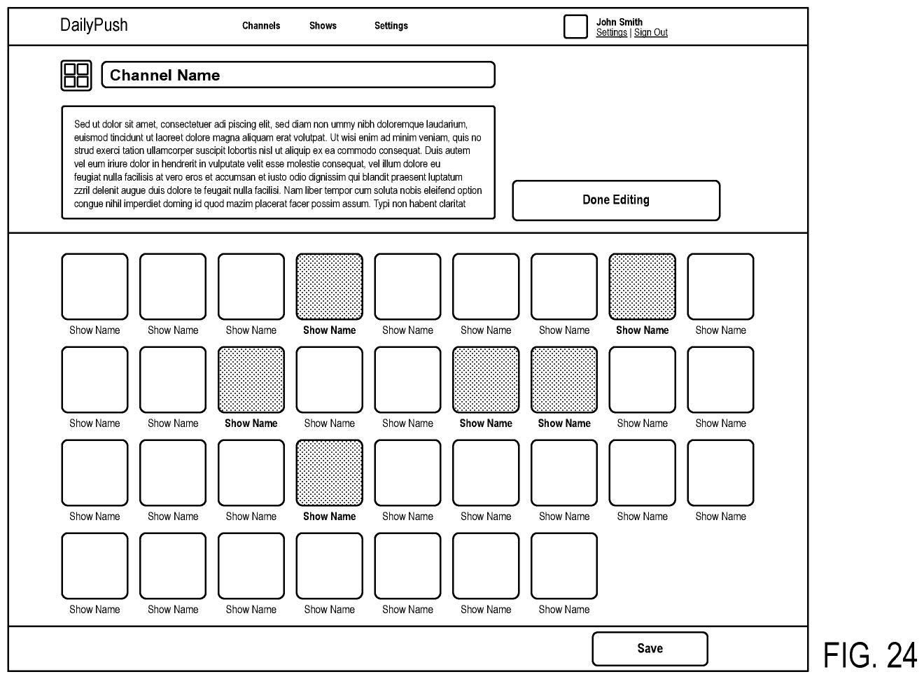

FIG. 24 shows an exemplary channel maintenance interface according to some embodiments. As shown, a user may view edit information about the channel, such as a name, a description, and videos associated with the channel.

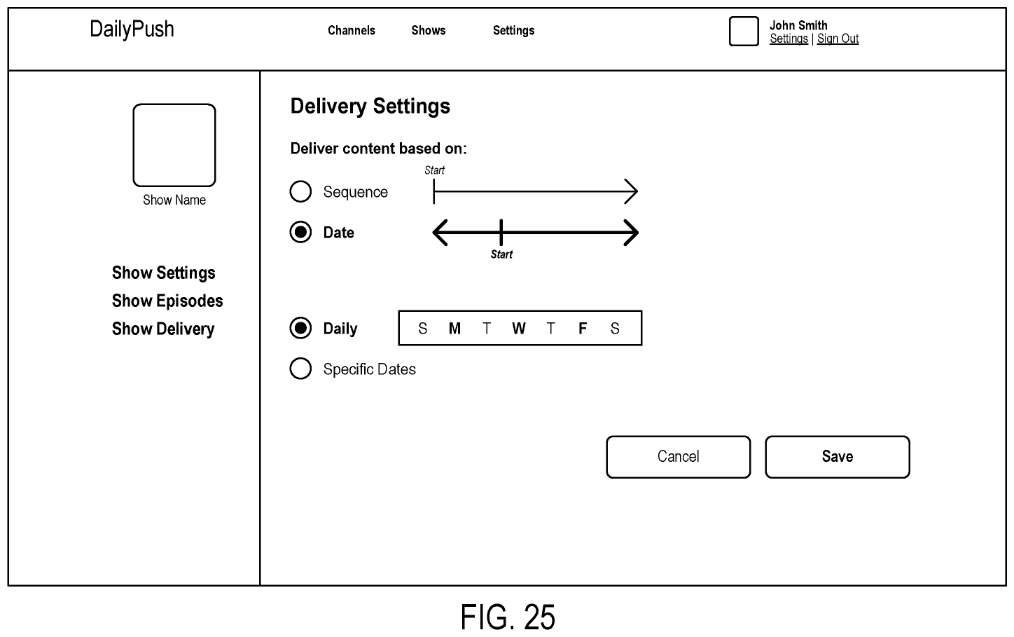

FIG. 25 shows an exemplary delivery settings interface according to some embodiments. As shown, a user may change the way that shows are delivered, such as by changing a delivery frequency and whether content is based on sequence or date.

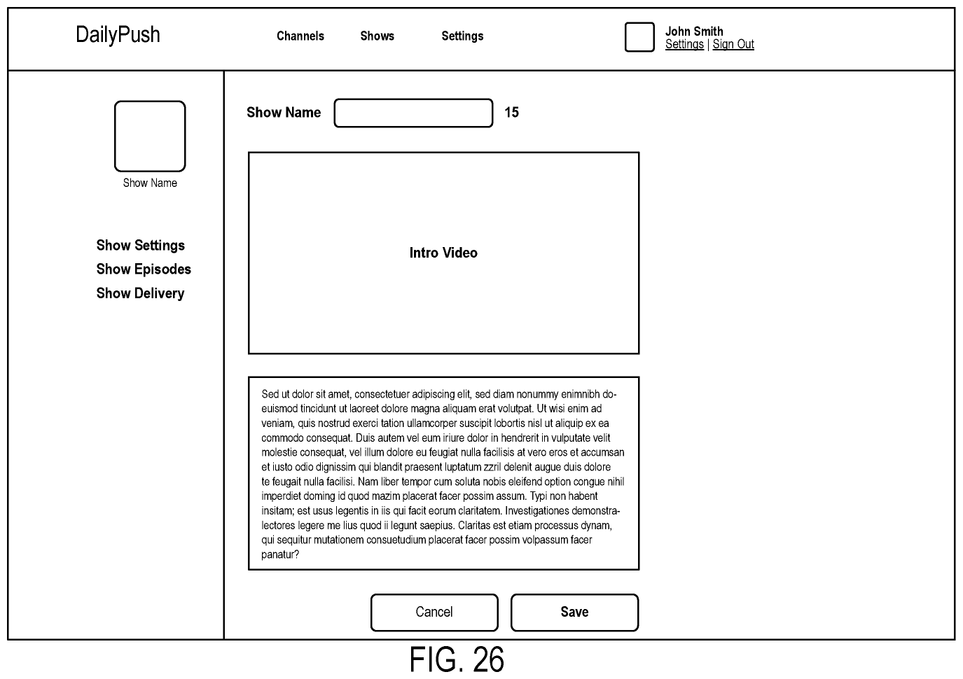

FIG. 26 shows an exemplary episode editing interface according to some embodiments. As shown, a user may change information about a show, such as a name and a description.

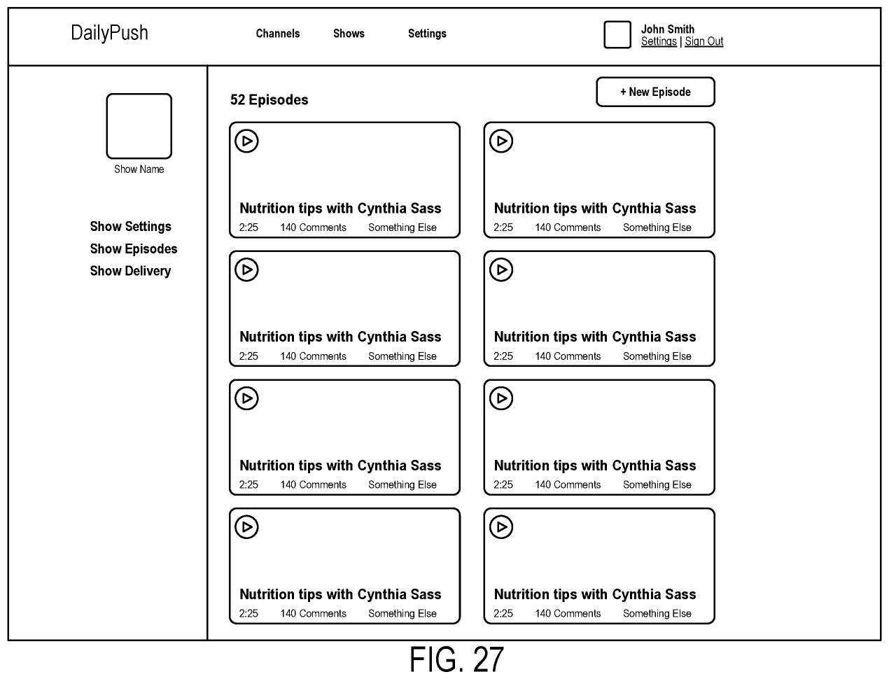

FIG. 27 shows an exemplary episode interface according to some embodiments. As shown, a user may view the episodes present on the system, select an episode, or add a new episode.

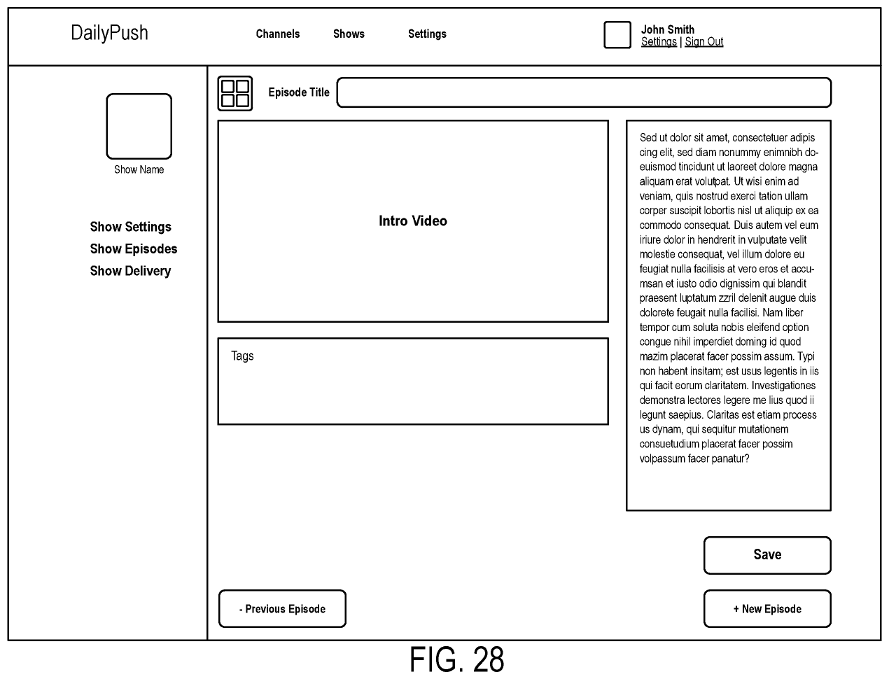

FIG. 28 shows an exemplary episode adding interface according to some embodiments. As shown, a user may add information about a show, such as a name, a description, and tags.

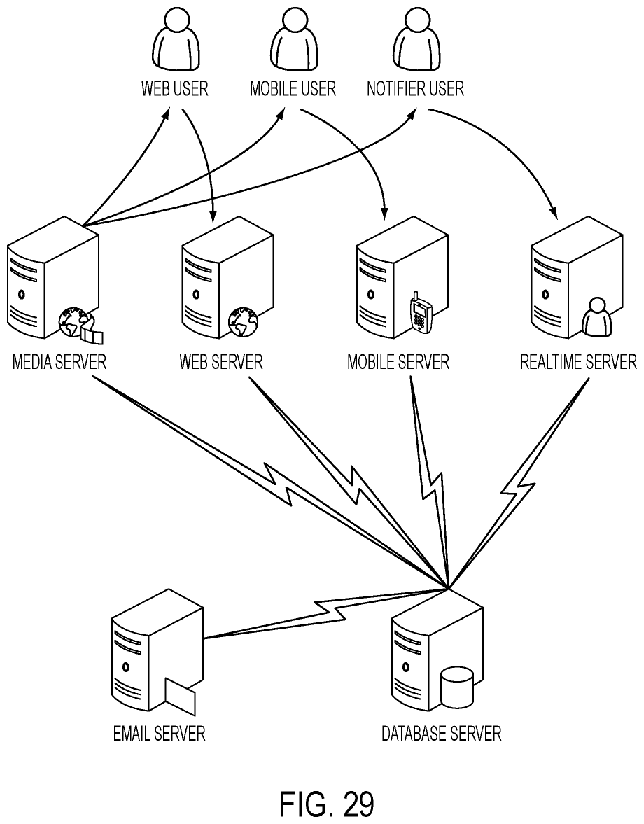

FIG. 29 shows an exemplary system diagram according to some embodiments. As shown, an exemplary system may contain a media server, a web server, a mobile server, a realtime server, database server, and an email server. Users of the system may include a web user, a mobile user, and a notifier user.

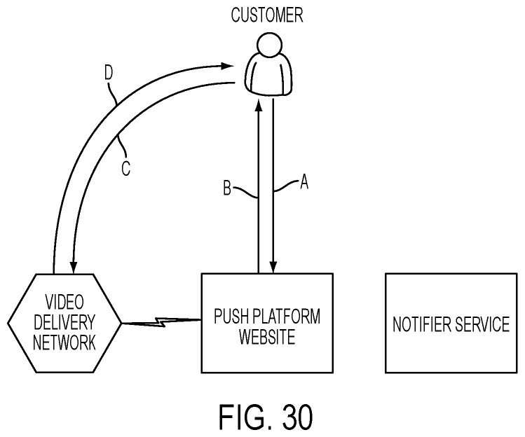

FIG. 30 shows an exemplary interaction diagram according to some embodiments. As shown, a customer first visits the push platform website. The customer then selects content to view. The customer is then directed to a URL for content at a content delivery network (“CDN”). The content delivery network then delivers the content to the customer.

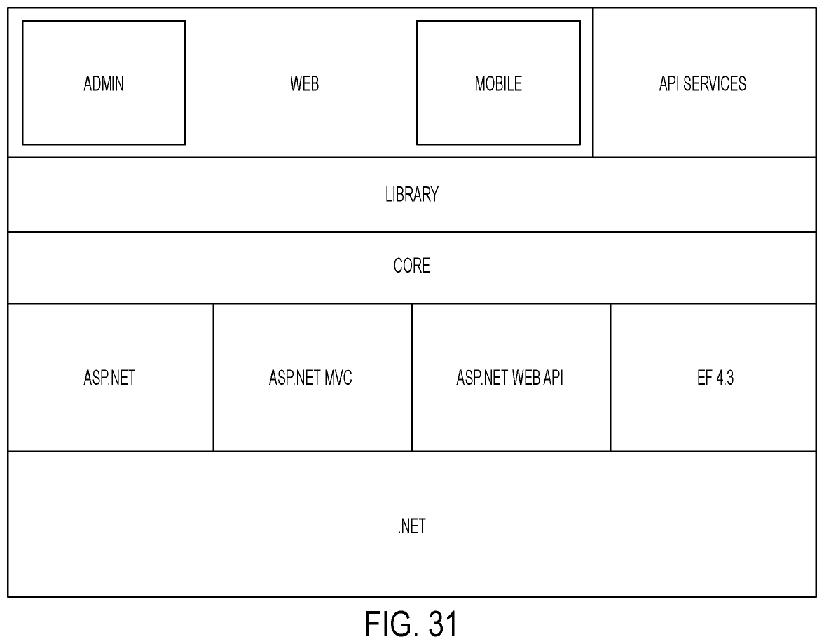

FIG. 31 shows an exemplary set of software modules according to some embodiments. As shown, the system may include an admin module, a web module, a mobile module, API services, a library module, a core module, ASP.NET components, ASP.NET MVC components, ASP.NET Web API components, EP components, and .NET components.

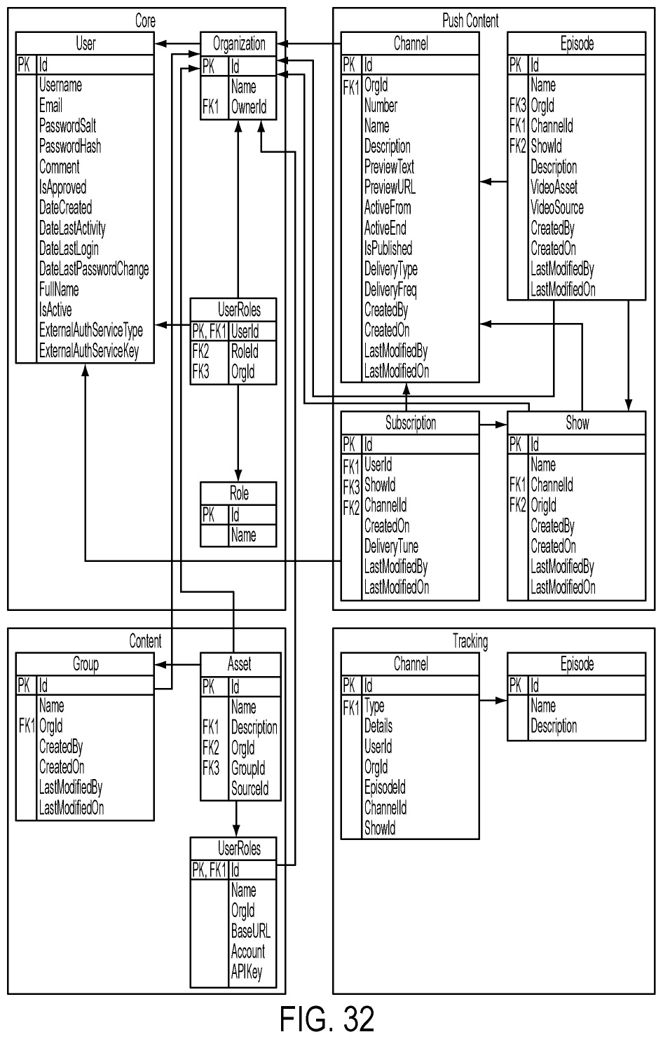

FIG. 32 shows a data model according to some embodiments. As shown, various data elements are organized into tables of a relational database model.

### Additional Notes

As noted, an exemplary system for implementing the overall system or portions thereof provided herein might include a general purpose computing device in the form of a computer, including a processing unit, a system memory, and a system bus that couples various system components including the system memory to the processing unit. The system memory may include read only memory (ROM) and random access memory (RAM). The computer may also include a magnetic hard disk drive for reading from and writing to a magnetic hard disk, a magnetic disk drive for reading from or writing to a removable magnetic disk, and an optical disk drive for reading from or writing to a removable optical disk such as a CD-ROM or other optical media. The drives and their associated machine-readable media provide nonvolatile storage of machine-executable instructions, data structures, program modules and other data for the computer.

Embodiments provided herein include program products comprising machine-readable media with machine-executable instructions or data structures stored thereon. Such machine-readable media can be any available storage media which can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to store desired program code in the form of machine-executable instructions or data structures and which can be accessed by a general purpose or special purpose computer or other machine with a processor. Combinations of the above are also included within the scope of machine-readable media. Machine-executable instructions comprise, for example, instructions and data which cause a general purpose computer, special purpose computer, or special purpose processing machines to perform a certain function or group of functions. Note that the machine-executable instructions/programming code may comprise algorithms embedded in Excel or other spreadsheets.

Embodiments provided herein have been described in the general context of method steps which may be implemented in embodiments by a program product including machine-executable instructions, such as program code, for example in the form of program modules executed by machines in networked environments. Generally, program modules include routines, programs, objects, components, data structures, etc., that perform particular tasks or implement particular data types. Multi-threaded applications may be used, for example, based on Java or C++. Machine-executable instructions, associated data structures, and program modules represent examples of program code for executing steps of the methods disclosed herein. The particular sequence of such executable instructions or associated data structures represent examples of corresponding acts for implementing the functions described in such steps.

Embodiments provided herein may be practiced with one or multiple computers in a networked environment using logical connections to one or more remote computers (including mobile devices) having processors. Logical connections may include a local area network (LAN) and a wide area network (WAN) that is presented here by way of example and not limitation. Such networked environments are commonplace in office-wide or enterprise-wide computer networks, and include intranets and the Internet, and may use a wide variety of different communication protocols. Those skilled in the art will appreciate that such network computing environments will typically encompass many types of computer system configurations, including personal computers, hand-held devices such as mobile phones and other PDA appliances, multi-processor systems, microprocessor-based or programmable consumer electronics, network PCs, minicomputers, mainframe computers, and the like. Embodiments provided herein may also be practiced in distributed computing environments where tasks are performed by local and remote processing devices that are linked (either by hardwired links, wireless links, or by a combination of hardwired and wireless links) through a communications network. In a distributed computing environment, program modules may be located in both local and remote memory storage devices.

It should be noted that although the flow charts provided herein show a specific order of method steps, it is understood that the order of these steps may differ from what is depicted. Also two or more steps may be performed concurrently or with partial concurrence. Such variation will depend on the software and hardware systems chosen and on designer choice. It is understood that all such variations are within the embodiments provided herein. Likewise, software and web implementations of the embodiments provided herein could be accomplished with programming techniques with rule based logic and other logic to accomplish the various database searching steps, correlation steps, comparison steps and decision steps. It should also be noted that the word “component” as used herein and in the claims is intended to encompass implementations using one or more lines of software code, and/or hardware implementations. It should also be noted that the phrase “a plurality” is intended to mean more than one, and is not intended to refer to any previous recitation of the word “plurality,” unless preceded by the word “the.”

All components, modes of communication, and/or processes described heretofore are interchangeable with similar components, modes of communication, and/or processes disclosed elsewhere in the specification, unless an express indication is made to the contrary.

While the embodiments provided herein has been described in conjunction with the exemplary embodiments outlined above, it is evident that many alternatives, modifications and variations will be apparent to those skilled in the art. Accordingly, the exemplary embodiments provided herein, as set forth above, are intended to be illustrative, not limiting. Various changes may be made without departing from the spirit and scope of the embodiments provided herein.

All literature and similar material cited in this application, including, but not limited to, patents, patent applications, articles, books, treatises, and web pages, regardless of the format of such literature and similar materials, are expressly incorporated by reference in their entirety. In the event that one or more of the incorporated literature and similar materials differs from or contradicts this application, including but not limited to defined terms, term usage, described techniques, or the like, this application controls.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

The above-described embodiments provided herein can be implemented in any of numerous ways. For example, some embodiments may be implemented using hardware, software or a combination thereof. When any aspect of an embodiment is implemented at least in part in software, the software code can be executed on any suitable processor or collection of processors, whether provided in a single computer or distributed among multiple computers.

In this respect, various aspects of the embodiments provided herein may be embodied at least in part as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium or non-transitory medium) encoded with one or more programs that, when executed on one or more computers or other processors, perform methods that implement the various embodiments of the technology discussed above. The computer readable medium or media can be transportable, such that the program or programs stored thereon can be loaded onto one or more different computers or other processors to implement various aspects of the present technology as discussed above.

The terms “program” or “software” are used herein in a generic sense to refer to any type of computer code or set of computer-executable instructions that can be employed to program a computer or other processor to implement various aspects of the present technology as discussed above. Additionally, it should be appreciated that according to one aspect of this embodiment, one or more computer programs that when executed perform methods of the present technology need not reside on a single computer or processor, but may be distributed in a modular fashion amongst a number of different computers or processors to implement various aspects of the present technology.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, the technology described herein may be embodied as a method, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” Any ranges cited herein are inclusive.

The terms “substantially” and “about” used throughout this Specification are used to describe and account for small fluctuations. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of” “only one of” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The claims should not be read as limited to the described order or elements unless stated to that effect. It should be understood that various changes in form and detail may be made by one of ordinary skill in the art without departing from the spirit and scope of the appended claims. All embodiments that come within the spirit and scope of the following claims and equivalents thereto are claimed.

What is claimed:

1. A method comprising:
receiving at one or more computing devices information about a user obtained at a point of interaction;
generating using the one or more computing devices, based at least in part on the information about the user, an individualized curriculum of engagement for the user; and
delivering using the one or more computing devices the individualized curriculum of engagement to the user.

2. The method of claim 1, further comprising:
scheduling using the one or more computing devices one or more interactions with the user prior to the step of delivering,
wherein the step of delivering is performed based on the step of scheduling.

3. The method of claim 1, wherein the step of generating further comprises selecting content from a content management source based at least in part on the point of interaction.

4. The method of claim 1, wherein in the step of generating, the individualized curriculum of engagement is further generated at least in part on an identity of an entity associated with the point of engagement.

5. The method of claim 1, further comprising:
generating, based at least in part on the information, a personalized URL for the user,
wherein the delivering further comprises sending the personalized URL to the user.

6. The method of claim 1, wherein the individualized curriculum of engagement comprises a single webpage with a plurality of embedded videos, and
wherein the embedded videos contain informational content related to at least one of a product and a service for the user at the point of interaction.

7. The method of claim 1, wherein the individualized curriculum of engagement comprises a single webpage with a plurality of embedded videos,
wherein the embedded videos contain informational content related to at least one of a product and a service for the user at the point of interaction, and
wherein the individualized curriculum of engagement further comprises access to a listing of other individualized curricula of engagement delivered to the user.

8. The method of claim 1, wherein the individualized curriculum of engagement comprises a single webpage with a plurality of embedded videos,
wherein the embedded videos contain informational content related to at least one of a product and a service for the user at the point of interaction, and
wherein the individualized curriculum of engagement further comprises access to promotional information for at least one of a product and a service related to the point of interaction.

9. The method of claim 1, wherein the individualized curriculum of engagement comprises a single webpage with a plurality of embedded videos,
wherein the embedded videos contain informational content related to at least one of a product and a service for the user at the point of interaction, and
wherein the individualized curriculum of engagement further comprises access to a live interaction interface that allows the user to receive information related to the point of interaction.

10. The method of claim 1, wherein the individualized curriculum of engagement comprises a plurality of videos,
wherein the plurality of videos contain informational content related to at least one of a product and a service for the user at the point of interaction, and
wherein the delivering further comprises individually delivering the plurality of videos to the user.

11. A method comprising:
receiving, using a computing device, information about a user at a point of interaction;
sending the information from the computing device to a webspace;
generating, based at least in part on the information in the webspace, an individualized curriculum of engagement for the user; and
delivering the individualized curriculum of engagement to the user.

12. The method of claim 10, further comprising:
securing the information at the computing device before the step of sending the information.

13. The method of claim 10, wherein the information comprises:
a name of the user;
an email address of the user; and
an identifier of a product related to the point of interaction for the user.

14. The method of claim 10, wherein the information comprises:
an identifier of the user;
an identifier of a drug prescribed to the user;
an identifier of an insurance provider for the user; and
an identifier of a pharmacy for the user.

15. The method of claim 10, further comprising:
generating, based at least in part on the information in the webspace, a personalized URL for the user,
wherein the delivering further comprises sending the personalized URL to the user.

16. A system for user engagement comprising:
an input computing device configured to receive information about a user at a point of interaction and to send the information to one or more servers;
the one or more servers configured to generate, based at least in part on the information received from the input computing device, an individualized curriculum of engagement for the user and to send the individualized curriculum of engagement to a user computing device; and
the user computing device configured to receive the individualized curriculum of engagement from the one or more servers.

17. The system of claim 16, wherein the user computing device is a smartphone belonging to the user, and
wherein the one or more servers are configured to send the individualized curriculum of engagement to the smartphone belonging to the user by sending a personalized URL in a text message.

18. The system of claim 16, wherein the user computing device is a smartphone belonging to the user, and
wherein the one or more servers are configured to send the individualized curriculum of engagement to the smartphone belonging to the user by sending a personalized URL in an email message.

19. The system of claim 16, wherein the one or more servers are configured to access a database of information regarding at least one of products and services related to the point of interaction in order to generate the individualized curriculum of engagement.

20. The system of claim 16, wherein the individualized curriculum of engagement comprises a single webpage with a plurality of embedded videos, and
wherein the embedded videos contain informational content related to a product or service for the user at the point of interaction.
